(19) United States
(12) Patent Application Publication
(10) Pub. No.: US 2023/0416400 A1
(43) Pub. Date: Dec. 28, 2023

(54) CHIMERIC ANTI-HUMAN BRAF V600E ANTIBODIES AND METHODS FOR MAKING AND USING THEM

(71) Applicant: Agilent Technologies, Inc., Santa Clara, CA (US)

(72) Inventors: Morten Draeby SORENSEN, Herlev (DK); Kirsten HOFF, Maaloev (DK)

(21) Appl. No.: 18/037,243

(22) PCT Filed: Nov. 15, 2021
(86) PCT No.: PCT/US2021/059368

§ 371 Date: May 16, 2023

Related U.S. Application Data
(60) Provisional application No. 63/114,123, filed on Nov. 16, 2020.

(51) Int. Cl. C07K 16/32; G01N 33/574

(52) U.S. Cl. CPC C07K 16/32; G01N 33/57419; C07K 2317/52; C07K 2317/55; C07K 2317/565

(57) ABSTRACT

In alternative embodiments, provided are chimeric or recombinant anti-human BRAF V600E antibodies, including products of manufacture and kits comprising them, and methods for making and using them, including for example their use in the detection or diagnosis of a cancer or other condition. In alternative embodiments, anti-BRAF V600E antibodies as provided herein are used together with an agent for determining whether MLH1 expression or activity is reduced or absent. In further alternative embodiments, anti-BRAF V600E antibodies as provided herein are used together with antibodies which specifically bind at least one protein selected from the group consisting of MLH1, PMS2, MSH2 and MSH6 to differentiate between Sporadic Colorectal Cancer (CRC) and probable Lynch syndrome.

### RELATED APPLICATIONS

This Patent Convention Treaty (PCT) International Patent Application claims the benefit of priority under 35 U.S.C. § 119(e) to U.S. Provisional Patent Application Ser. No. 63/114,123, Nov. 16, 2020. The aforementioned application is expressly incorporated herein by reference in their entirety and for all purposes

### TECHNICAL FIELD

This invention generally relates to immunohistochemistry (IHC) and cancer diagnosis. In alternative embodiments, provided are chimeric or recombinant anti-human BRAF V600E antibodies, including products of manufacture and kits comprising them, and methods for making and using them, including for example their use in the detection or diagnosis of a cancer or other conditions. In alternative embodiments, anti-BRAF V600E antibodies as provided herein are used together with an agent for determining whether MLH1 expression or activity is reduced or absent. In further alternative embodiments, anti-BRAF V600E antibodies as provided herein are used together with antibodies which specifically bind at least one protein selected from the group consisting of MLH1, PMS2, MSH2 and MSH6 to differentiate between Sporadic Colorectal Cancer (CRC) and probable Lynch syndrome.

### BACKGROUND

BRAF (also referred to as serine/threonine-protein kinase B-Raf, proto-oncogene B-Raf, v-Raf murine sarcoma viral oncogene homolog B) is a human gene that encodes a protein called B-Raf. The B-Raf protein is involved in directing cell growth by sending intracellular signals.

Mutation in the human BRAF gene has been associated with certain types of cancer. The most frequent mutation is where valine (V) is substituted by glutamic acid (E) at amino acid position 600 (called V600E). Oncogenic mutations in the kinase domain of the BRAF gene (such as BRAF V600E), results in constitutive activation of the B-raf protein which renders B-raf active in the absence of activating stimuli and can lead to excessive cell proliferation, apoptosis resistance and cancer. BRAF V600E is the result of a single point mutation (T1799A) in the kinase domain of the BRAF gene, which results in substitution of valine (V) to glutamic acid (E) at position 600 in the amino acid sequence of the B-raf protein.

The V600E mutation can be a driver mutation for melanoma, hairy cell leukemia, papillary thyroid carcinoma, colorectal cancer, non-small-cell lung cancer, Langerhans cell histiocytosis and ameloblastoma, and detection of the BRAF V600E mutation, for example, by immunohistochemistry (IHC), can aid in the diagnosis of these cancers.

However, antibodies targeting the human BRAF V600E mutation are very difficult to make due to only a single amino acid mutation needing to be differentiated by the antibody; this also makes their application in IHC problematic.

### SUMMARY

In alternative embodiments, provided are chimeric or recombinant anti-human BRAF V600E antibodies, including products of manufacture and kits comprising them, and methods for making and using them, including for example their use in the detection or diagnosis of a cancer.

In alternative embodiments, provided are chimeric or recombinant antibodies (Ab) or antigen binding proteins (ABP) capable of specifically binding a human BRAF V600E protein, selected from the group consisting of:
(a) a heterodimeric Ab or ABP (see FIG. 11A) comprising:
(i) a first and a second rabbit antibody Fc heavy chain, each comprising:
   (1) a C2 or a C3 (or CH2, CH3, respectively) rabbit antibody Fc heavy chain constant domain (see FIG. 11A, which illustrates these C2 and C3 domains, which are referenced as CH2 and CH3 in the figure), or portion thereof, of sufficient length to allow binding by an antibody capable of specifically binding said portion,
      wherein the first rabbit antibody Fc heavy chain constant domain, or a portion thereof, is bound to or associated with the second rabbit antibody Fc heavy chain constant domain, or a portion thereof,
   (2) a C1 (or CH1) rabbit antibody heavy chain constant domain or portion thereof (see FIG. 11A, which illustrates this C1 heavy chain domain, which is referenced as CH1 in the figure) capable of associating with a rabbit antibody light chain constant region bound to the C2 or C3 rabbit antibody Fc heavy chain constant domain or portion thereof, and
   (3) a heavy chain component or portion thereof of a mouse variable region antigen binding domain (ABD) (see FIG. 11A, which references this variable region as VH) or portion thereof capable of associating with a mouse light chain variable region ABD component or portion thereof, said heavy chain component of the mouse variable region antigen binding domain (ABD) or portion thereof being bound to the C1 rabbit antibody heavy chain constant domain or portion thereof,
   wherein when the heavy chain component of the mouse variable region ABD or portion thereof is associated with a light chain component of a mouse variable region ABD or portion thereof, the combined heavy chain variable region component or portion thereof and light chain variable region component or portion thereof together can specifically bind to the human BRAF V600E protein, and
(ii) two light chains, each comprising:
   (1) a C1 (or CL) rabbit antibody light chain constant region or portion thereof (see FIG. 11A, which illustrates the C1 light chain domain, which is referenced as CL in the figure),
   wherein the rabbit antibody light chain constant region, or a portion thereof, is associated with, or is bound to, a C1 rabbit antibody heavy chain constant domain or portion thereof, and
   (2) the light chain component of a mouse variable region antigen binding domain (ABD) (see FIG. 11A, which references this variable region as VL) or portion thereof in combination with, or is bound to or associated with, the mouse heavy chain variable region ABD component or portion thereof,
(b) a Fab heterodimer (see FIG. 11B and FIG. 11C) comprising:
   (i) a first chain comprising:
   (1) a C1 (or CH1, as referenced in FIG. 111B) rabbit antibody heavy chain constant domain or portion thereof capable of associating with or binding to a C1 (or CL) rabbit antibody light chain constant region or portion thereof, bound to:
   (2) a heavy chain component of a mouse variable region antigen binding domain (ABD) (referenced as VH in FIG. 111B) or portion thereof capable of associating with or binding to a mouse light chain variable region ABD component (referenced as VL in FIG. 111B) or portion thereof,
   wherein the C1 (or CH1) rabbit antibody light chain constant region or portion thereof is associated with, or is bound to, the C1 (or CL) rabbit antibody heavy chain constant domain; and
   (ii) a second chain comprising:
   (1) a C1 (or CL) rabbit antibody light chain constant region or portion thereof capable of associating with or binding to a C1 rabbit antibody heavy chain constant domain (or CH1) or portion thereof, bound to
   (2) a light chain component of a mouse variable region antigen binding domain (ABD) (referenced as VL in FIG. 111B) or portion thereof capable of associating with or binding to a mouse heavy chain variable region ABD component (referenced as VH in FIG. 111B) or portion thereof,
   wherein when the heavy chain component of the mouse variable region ABD is combined with, or is bound to, the light chain component of the mouse ABD, and the combined or bound heavy chain variable region ABD component and light chain variable region ABD component can specifically bind to the human BRAF V600E protein;
(c) a heterodimeric Ab or ABP comprising two Fab heterodimers of (b), wherein the C1 rabbit antibody heavy chain constant domain or portion thereof and the C1 rabbit antibody light chain constant region or portion thereof are joined by a linker (see FIG. 11C);
(d) a dimeric ABP (see FIG. 11D) comprising:
   (i) a first and a second heavy chain, each comprising:
      (1) a C2 or a C3 (or CH2, CH3, respectively) rabbit antibody Fc heavy chain constant domain, or a portion thereof of sufficient length to allow binding by an antibody capable of specifically binding said portion,
      wherein the C2 or a C3 rabbit antibody Fc heavy chain constant domain, or a portion thereof, of the first heavy chain is associated with or bound to the second C2 or C3 rabbit antibody Fc heavy chain constant domain, or a portion thereof,
      (2) a heavy chain component of a mouse variable region antigen binding domain (ABD) (referenced as VH in FIG. 11D) or portion thereof capable of associating with and bound to a mouse light chain variable region ABD component or portion thereof,
   wherein when the heavy chain component of the mouse variable region ABD or portion thereof is associated with and bound to the light chain component of the mouse variable region ABD or portion thereof, and the combined heavy chain variable component or portion thereof and light chain variable component or portion thereof can specifically bind to the human BRAF V600E protein, and
   (ii) a light chain comprising: the light chain component of a mouse variable region antigen binding domain (ABD) or portion thereof bound to the mouse heavy chain variable region ABD component or portion thereof, or
(e) a dimeric ABP (see FIG. 11E) comprising:
   (i) a first and a second heavy chain, each comprising:
      (1) a heavy chain component of a mouse variable region antigen binding domain (ABD) (referenced as VH in FIG. 11E) or portion thereof capable of specifically bind to the human BRAF V600E protein, or, a light chain component of a mouse variable region antigen binding domain (ABD) or portion thereof capable of specifically bind to the human BRAF V600E protein, and
      (2) a C2 or C3 (or CH2 or CH3, respectfully) rabbit antibody Fc heavy chain constant domain, or a portion thereof of sufficient length to allow binding by an antibody capable of specifically binding said portion,
      wherein the C2 or C3 rabbit antibody Fe heavy chain constant domain, or portion thereof, of the first heavy chain is associated with or bound to the other rabbit antibody C2 or C3 Fc heavy chain constant domain, or portion thereof of the second heavy chain.

In alternative embodiments of the chimeric or recombinant antibodies (Ab) or antigen binding proteins (ABP) as provided herein:
the first and the second rabbit antibody Fc heavy chain of embodiment (a)(i)(1) each comprises both a C2 rabbit antibody Fc heavy chain constant domain, or portion thereof, and a C3 rabbit antibody Fc heavy chain constant domain, or portion thereof;
in embodiment (a)(ii)(1) the first rabbit antibody Fc heavy chain constant domain, or portion thereof, is covalently or non-covalently bound to or associated with the second rabbit antibody Fc heavy chain constant domain, or portion thereof,
in embodiment (a)(ii)(2) the rabbit antibody light chain constant region, or portion thereof, is covalently or non-covalently associated with, or is bound to, the C1 rabbit antibody heavy chain constant domain;
in embodiment (b) the C1 rabbit antibody light chain constant region or portion thereof is covalently or non-covalently associated with, or is bound to, the C1 rabbit antibody heavy chain constant domain or portion thereof,
in embodiment (b) the light chain component of the mouse variable region antigen binding domain (ABD) or portion thereof is covalently or non-covalently capable of associating with or binding to a mouse heavy chain variable region ABD component or portion thereof,
in embodiment (c) the C1 rabbit antibody heavy chain constant domain or portion thereof and the C1 rabbit antibody light chain constant region or portion thereof are joined by a chemical or a peptide linker;
in embodiment (d) both the first and the second heavy chain each comprise both a C2 rabbit antibody Fc heavy chain constant domain, or portion thereof, and a C3 rabbit antibody Fc heavy chain constant domain, or portion thereof,
in embodiment (d) the C2 or the C3 rabbit antibody Fc heavy chain constant domain, or a portion thereof, of the first heavy chain is covalently or non-covalently associated with or bound to the second C2 or C3 rabbit antibody Fc heavy chain constant domain, or a portion thereof,
in embodiment (d) the light chain component of the mouse variable region antigen binding domain (ABD) or portion thereof is covalently or non-covalently bound to the mouse heavy chain variable region ABD component or portion thereof,
in embodiment (e) the first and the second heavy chain each comprises both a C2 and a C3 rabbit antibody Fc heavy chain constant domain, or a portion thereof of sufficient length to allow binding by an antibody capable of specifically binding said portion;
in embodiment (e) the C2 or C3 rabbit antibody Fc heavy chain constant domain, or portion thereof, of the first heavy chain is covalently or non-covalently associated with or bound to the rabbit antibody C2 or C3 Fc heavy chain constant domain, or portion thereof of the second heavy chain;
the rabbit antibody heavy chain constant region is a rabbit IgG antibody heavy chain constant region;
the rabbit antibody light chain constant region is a rabbit IgG antibody light chain constant region;
the rabbit antibody heavy chain constant region is a rabbit IgG antibody heavy chain constant region and the rabbit antibody light chain constant region is a rabbit IgG antibody light chain constant region;
the rabbit antibody light chain IgG constant region comprises a rabbit antibody kappa1, Kappa2, or a rabbit Lambda light chain constant region, and optionally the rabbit lambda light chain constant region comprises rabbit lambda light chain 1, 2, 3, 4, 5 or 6;
the heavy chain component of the mouse ABD comprises at least one of: a complementarity determining region (CDR) comprising SEQ ID NO:9 having one or more amino acid substitutions, additions (insertions) or deletions, a CDR comprising SEQ ID NO:10 having one or more amino acid substitutions, additions (insertions) or deletions, and a CDR comprising SEQ ID NO:11 having one or more amino acid substitutions, additions (insertions) or deletions, and optionally the chimeric or recombinant Ab or ABD is capable of specifically binding a human BRAF V600E protein;
the chimeric or recombinant Ab or ABD is capable of specifically binding a human BRAF V600E protein with greater or lesser affinity than the sequence without the at least one conservative amino acid substitution;
the heavy chain component of the mouse ABD comprises at least one of: a complementarity determining region (CDR) having an amino acid sequence having at least about 70%, 75%, 80%, 85%, 90%, 95%, 98% or complete sequence identity to SEQ ID NO:9, a CDR having an amino acid sequence having at least about 70%, 75%, 80%, 85%, 90%, 95%, 98% or complete sequence identity to SEQ ID NO:10,
and a CDR having an amino acid sequence having at least about 70%, 75%, 80%, 85%, 90%, 95%, 98% or complete sequence identity to SEQ ID NO:11,
and the chimeric or recombinant Ab or ABD is capable of specifically binding a human BRAF V600E protein;
the heavy chain component of the mouse ABD comprises SEQ ID NO:9, SEQ ID NO:10 and SEQ ID NO:11;
the light chain component of the mouse ABD comprises at least one of: a complementarity determining region (CDR) comprising SEQ ID NO:12 having one or more amino acid substitutions, additions (insertions) or deletions, a CDR comprising SEQ ID NO:13 having one or more amino acid substitutions, additions (insertions) or deletions and a CDR comprising SEQ ID NO:14 having one or more amino acid substitutions, additions (insertions) or deletions, and the chimeric or recombinant Ab or ABD is capable of specifically binding a human BRAF V600E protein;
the chimeric or recombinant Ab or ABD is capable of specifically binding a human BRAF V600E protein with greater or lesser affinity than the sequence without the at least one conservative amino acid substitution;
the light chain component of the mouse ABD comprises at least one of: a complementarity determining region (CDR) having an amino acid sequence having at least about 70%, 75%, 80%, 85%, 90%, 95%, 98% or complete sequence identity to SEQ ID NO:12, a CDR having an amino acid sequence having at least about 70%, 75%, 80%, 85%, 90%, 95%, 98% or complete sequence identity to SEQ ID NO:13, and a CDR having an amino acid sequence having at least about 70%, 75%, 80%, 85%, 90%, 95%, 98% or complete sequence identity to SEQ ID NO:14, and the chimeric or recombinant Ab or ABD is capable of specifically binding a human BRAF V600E protein;
the light chain component of the mouse ABD comprises SEQ ID NO:12, SEQ ID NO:13 and SEQ ID NO:14;
the chimeric or recombinant antibody comprises: a heavy chain comprising a sequence as set forth in SEQ ID NO:1 and a light chain as set forth in SEQ ID NO:5;
or, a heavy chain comprising a sequence as set forth in SEQ ID NO:2 and a light chain as set forth in SEQ ID NO:6.
each heavy chain comprises two rabbit Fc heavy chain constant region domains;
the linker comprises: a polyglycine linker, or a (GlyGlyGlySer)x (SEQ ID NO:15), wherein x is 2, 3 or 4;
the chimeric or recombinant antibody (Ab) or antigen binding protein (ABP) further comprises a heterologous protein or peptide, and optionally the heterologous protein or peptide comprises a peptide or polypeptide tag or detectable moiety, and optionally the peptide or polypeptide tag comprises an epitope tag, and optionally the epitope peptide or polypeptide tag comprises Myc-tag, a Human influenza hemagglutinin (HA) tag, FLAG-tag, GST-tag, a polyhistidine (His) or a 6×His (SEQ ID NO:16), V5-tag or OLLAS (E. coli OmpF Linker and mouse Langerin fusion Sequence); and/or
the chimeric or recombinant Ab or ABP is bound to a detectable agent or a binding moiety, and optionally the chimeric or recombinant Ab or ABP is covalently bound to the detectable agent or the binding moiety, and optionally the detectable agent or binding moiety comprises a biotin, a fluorescent or chemiluminescent label, a fluorophore, sulfoindocyanine, nile red, rhodamine, perylene, fluorenyl, coumarin, 7-methoxycoumarin (Mca), dabcyl, [2-(4-nitro-2,1,3-benzoxadiazol-7-yl)aminoethyl]trimethylammonium (NBD), Nile blue, Tamra, boron-dipyrromethene (BODIPY), or derivatives thereof, a dye, a radioisotope, a quantum dot or photoluminescent aqueous nanocrystal, a hapten, or an antibody binding epitope or domain; and optionally the fluorophore is or comprises a dansyl, a fluorescein, a carboxyfluorescein (FAM) or a 6-FAM moiety; and optionally the dye is or comprises a cyanine dye, a Cy3 or a Cy5; and optionally the hapten is or comprises a biotin, a theophylline, a digoxigenin, a carborane, a fluorescein or a bromodeoxyuridine moiety.

In alternative embodiments, provided are chimeric or recombinant nucleic acids encoding a chimeric or recombinant antibody as provided herein. In alternative embodiments the chimeric or recombinant nucleic acids further comprise and are operatively linked to a transcriptional regulatory element; and optionally the transcriptional regulatory element comprises a promoter; and optionally the promoter is a inducible promoter or a constitutive promoter.

In alternative embodiments, provided are expression cassettes, vectors, recombinant viruses, artificial chromosomes, cosmids, phagemids or plasmids comprising a chimeric or recombinant nucleic acid as provided herein.

In alternative embodiments, provided are cells comprising a chimeric or recombinant antibody as provided herein, a chimeric or recombinant nucleic acid as provided herein, or an expression cassette, vector, recombinant virus, artificial chromosome, cosmid, phagemid or plasmid as provided herein. In alternative embodiments, the cell is a bacterial, fungal, mammalian, yeast, insect or plant cell.

In alternative embodiments, provided are methods for detecting the presence of a human BRAF V600E protein in a cell or a tissue or organ or a portion of any of the foregoing comprising contacting the cell, tissue or organ or a portion of any of the foregoing with a chimeric or recombinant antibody as provided herein. In alternative embodiments of the methods:
the contacting comprises use of an immunohistochemistry (IHC) assay;
the method further comprises contacting the chimeric or recombinant antibody as provided herein, with a detectable agent to indicate or signal binding, or non-binding, of said chimeric or recombinant antibody to said BRAF V600E protein;
the detectable agent comprises an antibody or antigen binding moiety which binds to said rabbit constant region or portion thereof; and optionally the detectable agent comprises a chromogenic substrate bound to the antibody or antigen binding moiety, and optionally the chromogenic substrate comprises 3,3′-diaminobenzidine (DAB), 3,3′,5,5′-tetramethylbenzidine (TMB) or 2,2′-azino-bis(3-ethylbenzo-thiazoline-6-sulfonic acid) (ABTS);
the method further comprises the addition of a peroxidase enzyme and hydrogen peroxidase or equivalent oxidizing agent to oxidize the chromogenic substrate, and optionally the peroxidase comprises horse radish peroxidase; and/or
the method further comprises determining whether the cell, tissue, organ or portion of any of the foregoing has reduced mismatch repair gene MLH1 expression or activity or has no mismatch repair gene MLH1 expression or activity.

In alternative embodiments, provided are methods for determining whether a colorectal cancer (CRC) in a subject is the result of a sporadic mutation or probable Lynch syndrome comprising:
(a) determining whether the subject has reduced MLH1 expression or activity or has no MLH1 expression or activity; and
(b) if the subject has reduced MLH1 expression or activity or has no MLH1 expression or activity, determining whether the subject is expressing the BRAF V600E protein using a chimeric or recombinant antibody as provided herein,
wherein the colorectal cancer is determined to be the result of:
   (i) a sporadic mutation in the MLH1 gene if the subject expresses the BRAF V600E protein, or
   (ii) probable Lynch syndrome if the subject does not express the BRAF V600E protein.

In alternative embodiments, provided are methods for differentiating between Sporadic Colorectal Cancer (CRC) and Lynch syndrome in a CRC patient, or to identify probable Lynch syndrome in a CRC patient having loss of DNA mismatch repair gene MLH1, comprising conducting an immunohistochemistry assay using a chimeric or recombinant antibody as provided herein to detect the expression or presence of a human BRAF V600E protein in a cell or tissue sample from the CRC patient. In alternative embodiments of the methods:
the method comprises a protocol as set forth in FIG. 5; and/or
the method further comprises evaluating cell or a tissue using antibody which specifically binds to an MLH1 protein, and if the cell or tissue is found to be MLH1 deficient, the cell or tissue is evaluated for the presence of BRAF V600E protein, and if the cell or tissue is positive for staining for BRAF V600E protein the CRC is sporadic CRC, and if the cell or tissue if negative for staining for BRAF V600E protein the CRC is associated with Lynch syndrome.

In alternative embodiments, provided are methods for detecting or diagnosing a cancer, wherein the method comprises:
detection of expression or presence of a human BRAF V600E protein in a cell, tissue or organ sample, using a chimeric or recombinant antibody of any of claims 1 to 39, to detect the expression or presence of the human BRAF V600E protein in the cell, tissue or organ sample, and optionally the detection comprises conducting an immunohistochemistry (IHC) assay,
and detecting the expression or presence of the human BRAF V600E protein in the cell, tissue or organ sample detects or diagnoses the cancer.

In alternative embodiments of these methods:
the cancer is a colorectal cancer, a melanoma, non-Hodgkin lymphoma, hairy cell leukemia, papillary thyroid carcinoma, a lung cancer, Langerhans cell histiocytosis, ameloblastoma, a brain tumor, or hereditary nonpolyposis colorectal cancer (HNPCC), and optionally the melanoma is a malignant melanoma, and optionally the brain tumor is a glioblastoma or pleomorphic xanthoastrocytoma, and optionally the lung cancer is an adenocarcinoma of the lung or a non-small-cell lung cancer;
the method comprises or further comprises detection of expression or presence of a human BRAF V600E protein in a cell, tissue or organ sample from an individual in need thereof, and optionally the detection comprises conducting an immunohistochemistry (IHC) assay.

In alternative embodiments, provided are methods for treating, ameliorating or preventing a cancer comprising first detecting or diagnosing the cancer using a method as provided herein, followed by treatment of the individual in need thereof for the treatment, amelioration or prevention of the cancer.

In alternative embodiments, provided are methods for detecting or diagnosing an inflammatory disease, wherein the method comprises: detection of expression or presence of a human BRAF V600E protein in a cell, tissue or organ sample, using of chimeric or recombinant antibody as provided herein, to detect the expression or presence of the human BRAF V600E protein in the cell, tissue or organ sample; and detecting the expression or presence of the human BRAF V600E protein in the tissue or organ sample detects or diagnoses the inflammatory disease. In alternative embodiments, the inflammatory disease is Erdheim-Chester disease or polyostotic sclerosing histiocytosis. In alternative embodiments, the method comprises or further comprises detection of expression or presence of a human BRAF V600E protein in a cell, tissue or organ sample from an individual in need thereof. In alternative embodiments, the detection comprises conducting an immunohistochemistry (IHC) assay.

In alternative embodiments, provided are methods for treating, ameliorating or preventing an inflammatory disease comprising first detecting or diagnosing the inflammatory disease using a method as provided herein, followed by treatment of the individual in need thereof for the treatment, amelioration or prevention of the inflammatory disease.

In alternative embodiments, provided are uses of a chimeric or recombinant antibody as provided herein for detecting or diagnosing a cancer or an inflammatory disease. In alternative embodiments, the uses comprising use of an immunohistochemistry (IHC) assay.

In alternative embodiments, provided are kits comprising a chimeric or recombinant antibody as provided herein, and optionally the kit comprises components needed for an immunohistochemistry (IHC) assay, or comprises instructions for practicing a method as provided herein.

In alternative embodiments, provided are chimeric or recombinant antigen binding protein (ABP) capable of specifically binding a human BRAF V600E protein comprising: a rabbit antibody (Ab) heavy chain Fc region or portion thereof which is capable of specifically binding to a detectable secondary antibody or portion thereof; and, an antigen (Ag) binding region or an Ag-binding portion thereof comprising an Ab variable region from a species other than rabbit, wherein the variable region specifically binds to the human BRAF V600E protein. In alternative embodiments, of these chimeric or recombinant antigen binding proteins:
the antigen binding region comprises a Fab region;
the Fab region comprises a heavy chain region comprising a rabbit H C1 region fused to a mouse VH region and wherein said heavy chain region is associated with or bound to a light chain region comprising a rabbit L C1 region fused to a mouse VL region;
the antigen binding region comprises a ScFv region;
the ScFv region comprises a mouse VH region fused to a mouse VL region; and/or
the antigen binding region comprises a mouse VH region.

In alternative embodiments, provided are chimeric or recombinant antigen binding proteins comprising two Fab regions joined by a linker, wherein each Fab region comprises a heavy chain region comprising a rabbit H C1 region fused to a mouse VH region and wherein said heavy chain region is associated with a light chain region comprising a rabbit L C1 region fused to a mouse VL region.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1 illustrates an IHC analysis of a fully mouse version of an anti-human BRAF V600E antibody and an exemplary chimeric version of the antibody as provided herein (heavy chain SEQ ID NO:1 and light chain SEQ ID NO:2) in melanoma and colon carcinoma tissues, respectively.

FIG. 2 graphically illustrates binding kinetic measure between chimeric antibody (analyte) and peptide antigen (ligand) performed by Biolayer interferometry (BLI) using an Octet Red 96. The graph shows subtracted and aligned data for association and dissociation of 4 varying concentrations of chimeric antibody against the ligand, 33.3 (upper, or top line), 16.7 (line second from top), 8.33 (line second from bottom), and 2.78 nM (bottom, or lower line). The peptide is biotinylated and loaded on SAX sensors for the kinetic measurements.

FIG. 3 illustrates a table showing kinetic data from the exemplary chimeric version (FIG. 2) and mouse version of the antibody, respectively. For both measurements the same concentrations of the respective antibodies are used, together with identical buffers, measurement and analysis settings. The table include the KD value together with the on and off rates of the respective antibodies.

FIG. 4 schematically illustrates the role of BRAF V600E mutations in colorectal cancers and other cancers; image from J Transl Med. 2012 (see reference 1, below).

FIG. 5 schematically illustrates a Lynch syndrome diagnostic algorithm which uses the BRAF V600E antibody and a MMR (mismatch repair) IHC panel including antibodies which specifically recognize the MLH1, PMS2, MSH2 and MSH6 proteins; this algorithm can be used to differentiate between sporadic Sporadic Colorectal Cancer (CRC) and probable Lynch syndrome.

FIG. 6 illustrates the principle of the Dako FLEX++ system on the Dako OMNIS™ instrument, as used for TIC of the BRAF V600E antibody; the principle in the BRAF V600E FLEX++(Dako OMNIS™) IHC staining protocol is (as noted in the FIG. 6): 1. BRAF Antigen, 2. Primary antibody (an exemplary anti-BRAF antibody as provided herein), 3. Mouse Linker (or rabbit Linker), 4. Rabbit Linker (or mouse Linker), 5 ENVISION™ (or EnVision; a visualization reagent comprising an enzyme labeled polymer conjugated to secondary antibodies available from Agilent Technologies, Inc., Santa Clara, CA)), 6. DAB (visual brown reaction product at the antigen site).

FIG. 7 illustrates an IHC of Sporadic Colorectal Cancer (CRC) (block 4 BRAF+ stained with the exemplary chimeric BRAF V600E (45 ng/ml), where a weak to moderate cytoplasmic staining is observed in tumor cells. Note that a weak cytoplasmic background staining is observed with species-matched NCR.

FIG. 8 illustrates a melanoma tissue block stained with the exemplary chimeric BRAF V600E (45 ng/ml), where a weak to moderate cytoplasmic staining is observed in tumor cells.

FIG. 9 illustrates a papillary thyroid carcinoma stained with chimeric BRAF V600E (45 ng/ml), where a weak to moderate cytoplasmic staining is observed in tumor cells. Note that weak nuclear staining is observed in some tumor cells. Nuclear staining should be considered as non-specific staining.

FIG. 10 illustrates a hairy cell leukemia stained with chimeric BRAF V600E (45 ng/ml, where a weak to moderate cytoplasmic staining is observed in tumor cells. Comment: Artefacts, probably due to fixation, are present in this block.

FIG. 11 illustrates exemplary alternative antigen binding protein structures, and provided and described herein.

Like reference symbols in the various drawings indicate like elements.

### DETAILED DESCRIPTION

In alternative embodiments, provided are chimeric or recombinant anti-human BRAF V600E antibodies, including products of manufacture and kits comprising them, and methods for making and using them, including for example their use in the detection or diagnosis of a cancer such as: colorectal cancer (see ref 2, 3) melanoma (see ref. 2, 4, 5) (including malignant melanoma), non-Hodgkin lymphoma (see ref. 6), hairy cell leukemia (see ref. 7), papillary thyroid carcinoma (see ref. 8), adenocarcinoma of the lung (see ref. 9), non-small-cell lung cancer (NSCLC) (see ref. 4), Langerhans cell histiocytosis (see ref 10), ameloblastoma (see ref. 11), brain tumors including glioblastoma (see ref. 2, 12) and pleomorphic xanthoastrocytoma (see ref. 13), or hereditary nonpolyposis colorectal cancer (HNPCC) (see ref 6).

The BRAF mutation is most frequent in melanoma and thyroid cancers. Almost 60% of melanomas are reported to be BRAF mutant. In papillary thyroid carcinoma, 60% of cases show activating somatic alterations of genes encoding effectors in the MAPK signaling pathway, including BRAF. In colorectal cancer and NSCLC BRAF mutation has also been shown as a recurrent alteration.

Mutations of BRAF that lead to a constitutive activation of BRAF that is independent of upstream stimuli for growth and proliferation in cancer, such as BRAF V600E, has been under investigation for treatment with BRAF and/or MEK inhibitors.

Vemurafenib, one of the first BRAF inhibitors, is specific for BRAF V600E (see references 14 and 15). It was initially approved for advanced-stage melanoma treatment. In melanoma MEK inhibitors as single agents has been shown to be relatively modest, whereas the combination of BRAF and MEK inhibitors in BRAF-mutant melanomas has shown great success (see references 16 and 17).

In BRAF-mutant colorectal and thyroid cancers, RAF inhibitors have not show much clinical effect as single agents (see references 14 and 15).

In NSCLC clinical trial of dabrafenib and trametinib combination treatment achieved 64% objective response rate (see reference 18). The most common BRAF mutation in lung cancer is the BRAF V600E mutation, accounting for roughly 50% of BRAF-mutant NSCLC (see reference 19).

In metastatic colorectal cancer combination of BRAF and MEK inhibitors have achieved partial or complete response in a subset of patients (see reference 19). In a follow-up study, they also showed that combinatorial inactivation of BRAF, MEK, and EGFR achieves a higher response rate in BRAFV600E-positive patients (see reference 21). In Non-Hodgkin Lymphoma, Langerhans cell histiocytosis (see reference 22), glioblastoma (see references 23 and 24), ameloblastoma (see reference 25), and Hairy cell leukemia (see reference 26), pleomorphic xanthoastrocytoma (see reference 27) BRAF inhibitor alone or in combination with MEK inhibitors and or immunotherapy are in clinical trials or cases have been reported of treatment.

In alternative embodiments, chimeric or recombinant anti-human BRAF V600E antibodies as provided herein, including products of manufacture and kits comprising them, are used in the detection or diagnosis of an inflammatory disease such as erdheim-chester disease (see reference 28) (also called polyostotic sclerosing histiocytosis).

In alternative embodiments, chimeric or recombinant anti-human BRAF V600E antibodies as provided herein, including products of manufacture and kits comprising them, are used in the detection or diagnosis of Lynch syndrome, which is an autosomal dominant genetic condition that is associated with a high risk of colon cancer, endometrial cancer, or cancers of the ovary, stomach, small intestine, hepatobiliary tract, upper urinary tract, brain.

In alternative embodiments, provided are methods for treating, ameliorating or preventing a cancer such as: colorectal cancer, melanoma (including malignant melanoma), non-Hodgkin lymphoma, hairy cell leukemia, papillary thyroid carcinoma, adenocarcinoma of the lung, non-small-cell lung cancer, Langerhans cell histiocytosis, ameloblastoma, brain tumors including glioblastoma and pleomorphic xanthoastrocytoma, or hereditary nonpolyposis colorectal cancer (HNPCC), comprising use of chimeric or recombinant anti-human BRAF V600E antibodies as provided herein, including products of manufacture and kits comprising them, for the detection and/or diagnosis of the cancer, followed by the appropriate treatment or therapy, wherein optionally the appropriate treatment or therapy comprises administration of BRAF and/or MEK inhibitors.

In alternative embodiments, provided are methods for treating, ameliorating or preventing an inflammatory disease such as Erdheim-Chester disease (also called polyostotic sclerosing histiocytosis), comprising use of chimeric or recombinant anti-human BRAF V600E antibodies as provided herein, including products of manufacture and kits comprising them, for the detection and/or diagnosis of the inflammatory disease (for example, Erdheim-Chester disease), followed by the appropriate treatment or therapy, wherein optionally the appropriate treatment or therapy comprises administration of BRAF and/or MEK inhibitors.

In alternative embodiments, provided are methods for treating, ameliorating or preventing Lynch syndrome, including the possibly associated colon cancer, endometrial cancer, or cancers of the ovary, stomach, small intestine, hepatobiliary tract, upper urinary tract, brain, comprising use of chimeric or recombinant anti-human BRAF V600E antibodies as provided herein, including products of manufacture and kits comprising them, for the detection and/or diagnosis of the Lynch syndrome or associated cancer, followed by the appropriate treatment or therapy, wherein optionally the appropriate treatment or therapy comprises administration of BRAF and/or MEK inhibitors.

### Differentiating Between Sporadic Colorectal Cancer (CRC) and Lynch Syndrome

In alternative embodiments, exemplary anti-human BRAF V600E antibodies as provided herein are used to differentiate between sporadic colorectal cancer (CRC) and probable Lynch syndrome.

Lynch syndrome is an autosomal dominant genetic condition the causes a high risk for CRC as well as cancers in other sites. Lynch syndrome accounts for 2% to 5% of all CRCs. Lynch syndrome pertains to those individuals in whom a germline mutation in one of the DNA mismatch repair (MMR) genes (MLH1, MSH2, MSH6 or PMS2) has been identified.

In Lynch syndrome immunohistochemistry (IHC) can be used to show abnormal MMR (MLH1, MSH2, MSH6 and/or PMS2) protein expression (a MMR (MLH1, PMS2, MSH2 and MSH6) IHC panel can be used). Lynch syndrome sufferers inherit one germline mutant allele and one normal, wild type allele of MLH1, MSH2, MSH6 or PMS2. During tumor formation, the normal allele is inactivated by mutation or loss, leading to no expression of functional alleles. MMR (MLH1, MSH2, MSH6 and PMS2) abnormal expression in Lynch tumors may be observed in IHC in two staining patterns: either complete loss of expression of MMR protein or a patchy/weak expression depending on the effect on protein expression of a given mutation.

However, loss of MLH1 protein can also be due to hypermethylation of the promotor in the MLH1 gene and indicates a sporadic occurrence of CRC compared to Lynch syndrome. In addition, the presence of BRAF V600E is close correlated with hypermethylation in the promoter of the MLH1 gene in sporadic CRC (see references 1, 2, 3).

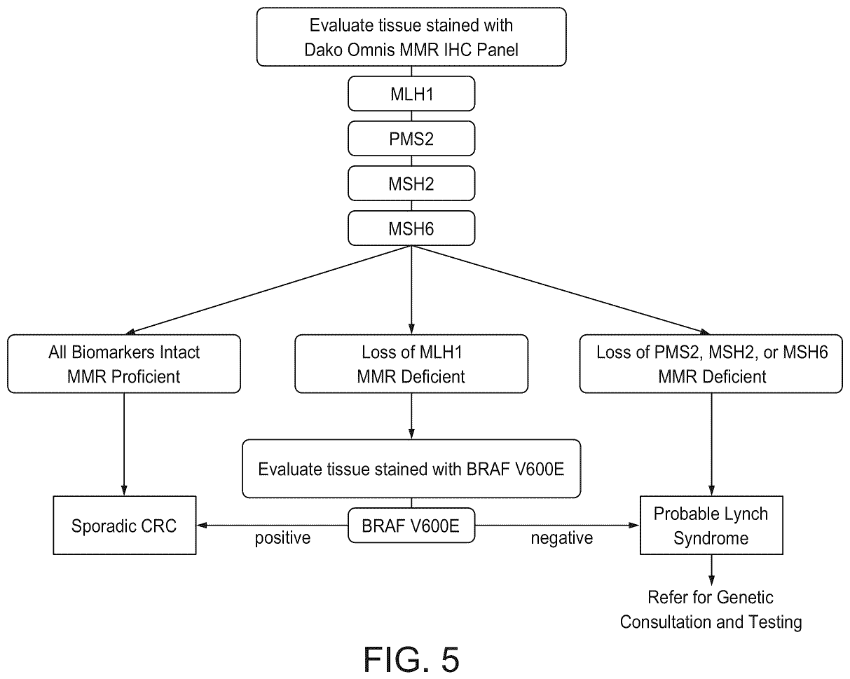

Thus, a MMR (MLH1, MSH2, MSH6 and/or PMS2) IHC panel can be used to identify probable Lynch syndrome in CRC patients and, in cases with loss of MLH1, a negative BRAF V600E IHC test result indicates probable Lynch syndrome. A positive BRAF V600E IHC test result indicates sporadic CRC (non-Lynch syndrome). As illustrated in FIG. 5, showing a Lynch syndrome diagnostic algorithm, the BRAF V600E antibody and the MMR IHC panel can be used to differentiate between sporadic CRC and probable Lynch syndrome. In CRC cases with loss of MLH1, a negative BRAF V600E test result indicates probable Lynch syndrome. A positive BRAF V600E test result indicates sporadic CRC (non-Lynch syndrome).

### Exemplary Mechanisms of Action

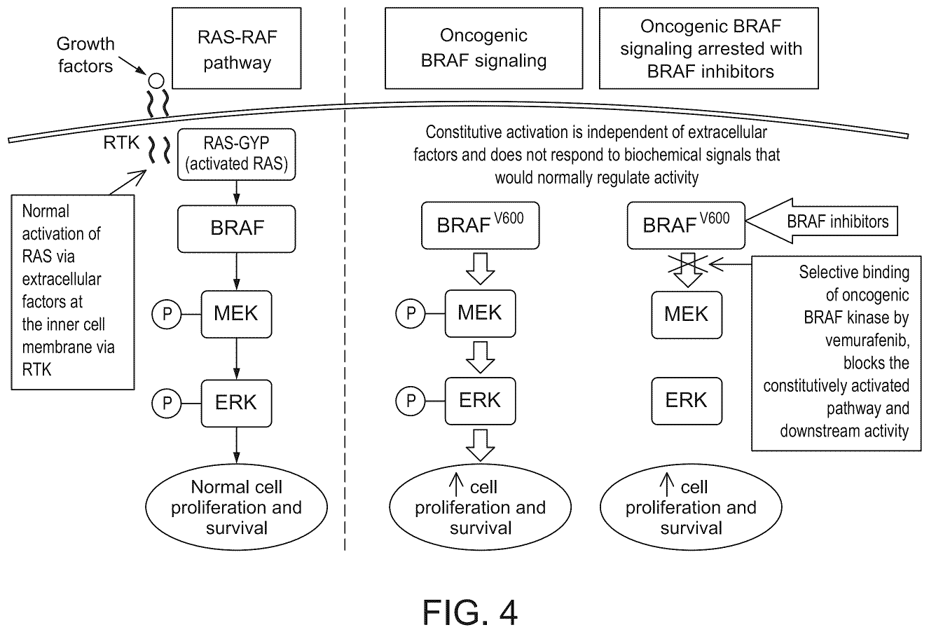

While the invention is not limited by any particular mechanism of action, the presence of the V600E mutation in melanoma is directly linked to a treatment. BRAF is a serine/threonine protein kinase activating the MAP kinase/ERK-signaling pathway, as illustrated in FIG. 4. About 50% of melanomas harbors activating BRAF mutations, and over 90% are the V600E mutation. BRAF V600E has been implicated in different mechanisms underlying melanomagenesis, most of which are due to the deregulated activation of the downstream MEK/ERK effectors. Targeted therapy with BRAF and MEK inhibitors is associated with significant long-term treatment benefit in patients with BRAF V600-mutated melanoma. BRAF inhibitors are drugs that attack the BRAF protein directly, such as vemurafenib (e.g., ZELBORAF™) dabrafenib (e.g., TAFINLAR™), and encorafenib (e.g., BRAFTOVI™). The MEK gene works together with the BRAF gene, so drugs that block MEK proteins can also help treat melanomas with BRAF gene changes. MEK inhibitors include trametinib (e.g., MEKINIST™), cobimetinib (e.g., COTELLIC™), and binimetinib (e.g., MEKTOVI™).

### Use of Exemplary Antibodies Result in Improved IHC Results

In alternative embodiments, use of chimeric or recombinant anti-human BRAF V600E antibodies as provided herein in immunohistochemistry (IHC) protocols results in improved results. In IHC, a primary antibody (IgG) is used to specifically bind a protein of interest (for example, BRAF V600E) in a tissue sample, for example, a Formalin-Fixed Paraffin-Embedded (FFPE) tissue, or equivalent. Visualization of this primary antibody (Ab) in the tissue sample, e.g., the FFPE tissue, can be made using a secondary antibody (Ab) or other polypeptide capable of specifically binding the primary Ab (for example, BRAF V600E). Frequently, the IHC primary Ab is an IgG immunoglobulin, for example, a mouse IgG immunoglobulin, where the IgG can be one of the IgG or IgM isotypes, for example, a mouse IgG1, IgG2a, IgG2b, IgG3 and IgM. In alternative embodiments, for increased effectiveness in detecting a mix of mouse IgG isotypes, the secondary Ab is or comprises a polyclonal anti-mouse IgG and/or IgM antibody, and the secondary Ab can be a mixture of Abs specific towards the constant region of each of the primary antibody isotypes such that one secondary Ab reagent (for example, a polyclonal serum) can be used for detecting all the primary Abs. The secondary Ab can be any mixture of IgG isotypes, and can also include IgM isotype Abs.

In alternative embodiments, provided is an improved primary Ab in the form of a chimeric or recombinant anti-human BRAF V600E Ab where the constant regions of the Ab are exchanged with that of another species, for example, in one embodiment the mouse constant regions are replaced with rabbit constant regions. This allows for usage of a secondary Ab raised against (or specific for) the constant region of a different species than that of the primary Ab, for example, rabbit in place of mouse. Rabbits have only one isotype of IgG in contrast to the several IgG isotypes in mouse. Thus, in some embodiments, the chimeric or recombinant antibody contains a constant region from a species that has only one isotype of IgG. This constant region shift can be an advantage for multiplexing (where more than one primary antibody is used, for example, from different species) such as double staining with different colors to specifically look at co-localization of multiple markers, for example, different primary antibodies.

The chimeric or recombinant anti-human BRAF V600E Ab as provided herein has resulted in a great reduction of the antibody titer which provides optimal performance in IHC. For example, in alternative embodiments, if the mouse antibody which provides optimal performance is titrated to 500 ng/mL, the chimeric or recombinant antibody as provided herein can be titrated to between about 30 ng/mL to 62.5 ng/mL depending on method of antibody quantification; thus, use of an exemplary chimeric or recombinant anti-human BRAF V600E Ab in IHC results in an about 8 to 16-fold reduction of antibody needed in the assay. The diagnostic usage of the exemplary chimeric or recombinant anti-human BRAF V600E Ab as provided herein is at least as good, if not better, as the mouse version of the antibody.

### Sequences of Exemplary Chimeric or Recombinant Anti-Human BRAF V600E Abs

An exemplary sequence of an exemplary chimeric or recombinant anti-human BRAF V600E Ab as provided herein is (the complementarity determining regions, or CDRs, are bolded):

Heavy chain with signal peptide:
(SEQ ID NO: 1)
METGLRWLLLVAVLKGVQCQAYLQQSGAELVRPGASVKMSCKASGYTFT
SYNMHWIKQTPRQGLEWIGAIYPGNGDTSYNQKFKGKATLTVDRSSNTA
YMQLSSLTSEDSAVYFCARDGISPWGQGTLVTVSSGQPKAPSVFPLAPC
CGDTPSSTVTLGCLVKGYLPEPVTVTWNSGTLTNGVRTFPSVRQSSGLY
SLSSVVSVTSSSQPVTCNVAHPATNTKVDKTVAPSTCSKPTCPPPELLG
GPSVFIFPPKPKDTLMISRTPEVTCVVVDVSQDDPEVQFTWYINNEQVR
TARPPLREQQFNSTIRVVSTLPIAHQDWLRGKEFKCKVHNKALPAPIEK
TISKARGQPLEPKVYTMGPPREELSSRSVSLTCMINGFYPSDISVEWEK
NGKAEDNYKTTPAVLDSDGSYFLYSKLSVPTSEWQRGDVFTCSVMHEAL
HNHYTQKSISRSPGK
Heavy chain without signal peptide:
(SEQ ID NO: 2)
QAYLQQSGAELVRPGASVKMSCKASGYTFTSYNMHWIKQTPRQGLEWIG
AIYPGNGDTSYNQKFKGKATLTVDRSSNTAYMQLSSLTSEDSAVYFCAR
DGISPWGQGTLVTVSSGQPKAPSVFPLAPCCGDTPSSTVTLGCLVKGYL
PEPVTVTWNSGTLTNGVRTFPSVRQSSGLYSLSSVVSVTSSSQPVTCNV
AHPATNTKVDKTVAPSTCSKPTCPPPELLGGPSVFIFPPKPKDTLMISR
TPEVTCVVVDVSQDDPEVQFTWYINNEQVRTARPPLREQQFNSTIRVVS
TLPIAHQDWLRGKEFKCKVHNKALPAPIEKTISKARGQPLEPKVYTMGP
PREELSSRSVSLTCMINGFYPSDISVEWEKNGKAEDNYKTTPAVLDSDG
SYFLYSKLSVPTSEWQRGDVFTCSVMHEALHNHYTQKSISRSPGK
(SEQ ID NO: 4)
GQPKAPSVFPLAPCCGDTPSSTVTLGCLVKGYLPEPVTVTWNSGTLTNG
VRTFPSVRQSSGLYSLSSVVSVTSSSQPVTCNVAHPATNTKVDKTVAPS
TCSKPTCPPPELLGGPSVFIFPPKPKDTLMISRTPEVTCVVVDVSQDDP
EVQFTWYINNEQVRTARPPLREQQFNSTIRVVSTLPIAHQDWLRGKEFK
CKVHNKALPAPIEKTISKARGQPLEPKVYTMGPPREELSSRSVSLTCMI
NGFYPSDISVEWEKNGKAEDNYKTTPAVLDSDGSYFLYSKLSVPTSEWQ
RGDVFTCSVMHEALHNHYTQKSISRSPGK

where METGLRWLLLVAVLKGVQC (SEQ ID NO:3) is the rabbit IgG heavy chain signal peptide; and

the rabbit IgG heavy chain constant domain is

Summary heavy chain: Amino acids (aa) 1 to 19=signal peptide; aa 20 to 133=mouse heavy chain variable domain; aa 134 to 456=Rabbit heavy chain IgG constant domains. CDR regions according to IMGT numbering: CDR1 aa 45 to 52 (GYTFTSYN) (SEQ ID NO:9), CDR2 aa 70 to 77 (IYPGNGDT) (SEQ ID NO:10), and CDR3 aa 116 to 122 (ARDGISP) (SEQ ID NO:11).

Light chain with signal peptide:
(SEQ ID NO: 5)
MDTRAPTQLLGLLLLWLPGATFDIKMTQSPSSMYASLGERVTITCKASQ
DINRYLSWFQQKPGKSPKTLIYRANRVLDGVPSRFSGSGSGQDYSLTIS
SLESEDMGIYYCLHYDEYPYTYGGGTKLEIKGDPVAPTVLIFPPAADQV
ATGTVTIVCVANKYFPDVTVTWEVDGTTQTTGIENSKTPQNSADCTYNL
SSTLTLTSTQYNSHKEYTCKVTQGTTSVVQSFNRGDC
Light chain without signal peptide:
(SEQ ID NO: 6)
DIKMTQSPSSMYASLGERVTITCKASQDINRYLSWFQQKPGKSPKTLIY
RANRVLDGVPSRFSGSGSGQDYSLTISSLESEDMGIYYCLHYDEYPYTY
GGGTKLEIKGDPVAPTVLIFPPAADQVATGTVTIVCVANKYFPDVTVTW
EVDGTTQTTGIENSKTPQNSADCTYNLSSTLTLTSTQYNSHKEYTCKVT
QGTTSVVQSFNRGDC

where MDTRAPTQLLGLLLLWLPGATF (SEQ ID NO:7) is the rabbit IgG heavy chain signal peptide; and

the rabbit kappa1 light chain constant domain is:

(SEQ ID NO: 8)
GDPVAPTVLIFPPAADQVATGTVTIVCVANKYFPDVTVTWEVDGTTQTT
GIENSKTPQNSADCTYNLSSTLTLTSTQYNSHKEYTCKVTQGTTSVVQS
FNRGDC

Summary light chain: Amino acids (aa) 1 to 22=Signal peptide, aa 23 to 129=mouse light chain variable domain; aa 130 to 233=rabbit Kappa1 constant domain.

CDR regions according to IMGT numbering: CDR1 aa 49 to 54 (QDINRY) (SEQ ID NO:12), CDR2 aa 72 to 74 (RAN) (SEQ ID NO:13), and CDR3 aa 111 to 119 (LHYDEYPYT) (SEQ ID NO:14).

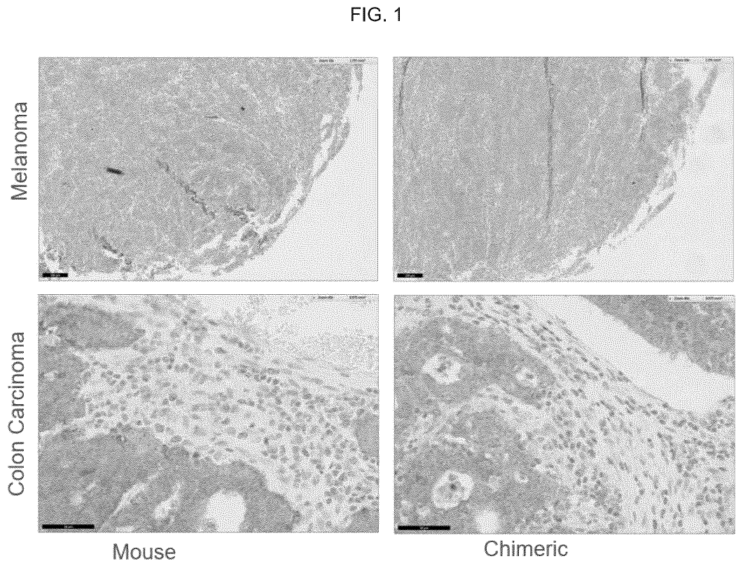

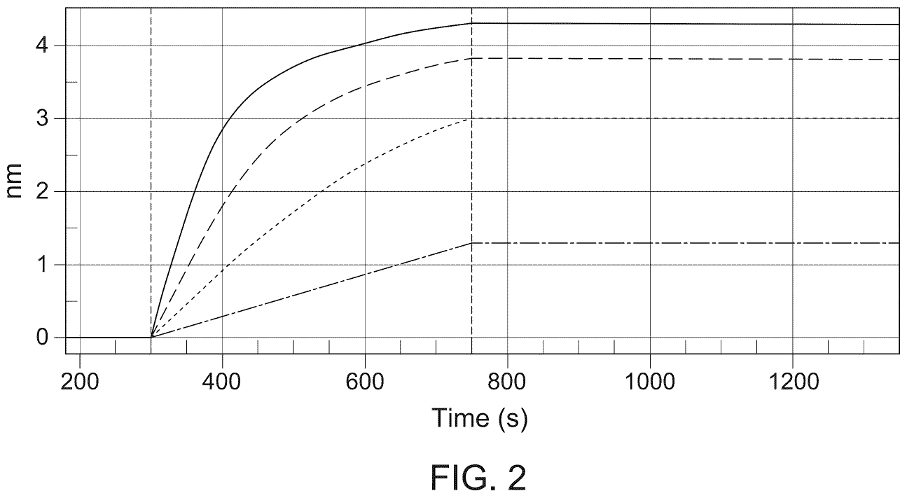

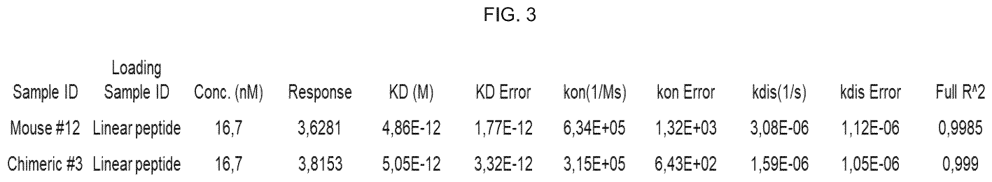

FIG. 1 illustrates an IHC of a mouse version of the antibody (Ab) and an exemplary chimeric version of the Ab as provided herein, or a recombinant Ab comprising heavy chain SEQ ID NO:2 and light chain SEQ ID NO:6, from a testing of the Ab's performance in an IHC of melanoma and colon carcinoma cells. FIG. 2 illustrates the kinetic binding curves of this antibody using a synthetic peptide as ligand, including the amino acids of and surrounding the BRAF V600E mutation. FIG. 3 illustrates a table summarizing the data from FIG. 2, including the KD value of the mouse version of the Ab and the exemplary chimeric version of the Ab, where FIG. 2 illustrates kinetic binding curves of the chimeric antibody (SEQ ID NO:2/SEQ ID NO:6). Kinetic binding curves were obtained from Octet Red96 (Ab concentration, or [Ab], range is between 2.78 nM and 33.3 nM). Ligand: BRAF V600E peptide, Analyte: Chimeric Antibody. The KD value of the mouse and chimeric versions are very similar, though the chimeric Ab tends to have a slightly slower on rate as well as off rate (as compared to the all mouse Ab.

### Expression of Recombinant Chimeric Antibodies

In alternative embodiments, chimeric or recombinant Abs as provided herein, including the exemplary chimeric or recombinant anti-human BRAF V600E Ab comprising heavy chain SEQ ID NO:1 and light chain SEQ ID NO:5, with signal peptide, or without the signal peptide (SEQ ID NO:2 and SEQ ID NO:6)) can be expressed as a recombinant Ab using a plasmid (or any expression vehicle) encoding the respective heavy and light chains, or the heavy chain and the light chain can be encoded in separate expression vehicles. In some embodiments, the heavy and light chains can be (cis- or trans-) expressed from a pTT5™ vector(s) (National Research Council Canada, NRC-CNRC, Canada) in HEK293-6E cells. In alternative embodiment, the vector or vectors expressing the heavy and/or light chains are episomal or are chromosomally integrated, for example, in a stable cell line capable of synthesizing, optionally inducibly synthesizing, the heavy and/or light chains.

In alternative embodiments, provided are nucleic acids encoding chimeric or recombinant Abs as provided herein. Nucleic acids as provided herein can be made, isolated and/or manipulated by, for example, cloning and expression of cDNA libraries, amplification of message or genomic DNA by PCR, and the like. Nucleic acids used to practice embodiments as provided herein, whether RNA, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, fungal, mammalian, yeast, insect or plant cell expression systems.

Alternatively, these nucleic acids can be synthesized in vitro by well-known chemical synthesis techniques, as described in, for example, Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, for example, Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice embodiments as provided herein comprises screening and re-cloning inserts isolated or amplified from, e.g., genomic clones or cDNA clones. Sources of nucleic acids include recombinant nucleic acid sequences, genomic or cDNA libraries contained and/or expressed in, for example, mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages, phagemids or plasmids.

In alternative embodiments, nucleic acids as provided herein are operably linked to transcriptional regulatory elements, including promoters, with can be constitutive or inducible transcriptional regulatory elements.

In alternative aspects, provided are “expression cassettes” comprising a nucleotide sequence as provided herein, for example encoding a chimeric or recombinant antibody as provided herein. Expression cassettes can include at least a transcriptional regulatory element, for example, a promoter, operably linked with an antibody coding sequence, and optionally can also include transcription termination signals. Additional factors necessary or helpful in effecting expression may also be used, for example, enhancers.

In alternative aspects, expression cassettes used to practice embodiments as provided herein include plasmids, expression vectors, recombinant viruses, any form of recombinant “naked DNA” vector, and the like. In alternative aspects, a “vector” used to practice embodiments as provided herein can comprise a nucleic acid that can infect, transfect, transiently or permanently transduce a cell. In alternative aspects, a vector used to practice embodiments as provided herein can be a naked nucleic acid, or a nucleic acid complexed with protein or lipid. In alternative aspects, vectors used to practice embodiments as provided herein can comprise viral or bacterial nucleic acids and/or proteins, and/or membranes (e.g., a cell membrane, a viral lipid envelope, etc.). In alternative aspects, vectors used to practice embodiments as provided herein can include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and can include both the expression and non-expression plasmids. In alternative aspects, the vector used to practice embodiments as provided herein can be stably replicated by the cells during mitosis as an autonomous structure, or can be incorporated within the host's genome.

In alternative aspects, “promoters” used to practice embodiments as provided herein include all sequences capable of driving transcription of a coding sequence in a cell, for example, a bacterial, yeast, fungal, plant, insect (e.g., baculovirus) or mammalian cell. Thus, promoters used in the constructs include cis-acting transcriptional control elements and regulatory sequences that are involved in regulating or modulating the timing and/or rate of transcription of a gene. For example, a promoter used to practice embodiments as provided herein can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences can interact with proteins or other biomolecules to carry out (turn on/off, regulate, modulate, etc.) transcription.

“Constitutive” promoters used to practice embodiments as provided herein can be those that drive expression continuously under most environmental conditions and states of development or cell differentiation. “Inducible” or “regulatable” promoters used to practice embodiments as provided herein can direct expression of a nucleic acid as provided herein under the influence of environmental conditions or developmental conditions. Examples of environmental conditions that may affect transcription by inducible promoters used to practice embodiments as provided herein include the presence of an inducing factor administered to a cell.

In alternative embodiments, peptides and polypeptides used to practice embodiments as provided herein can comprise any “mimetic” and/or “peptidomimetic” form. In alternative embodiments, peptides and polypeptides used to practice embodiments as provided herein can comprise synthetic chemical compounds which have substantially the same structural and/or functional characteristics of the natural polypeptide, for example, a chimeric or recombinant antibody as provided herein. The mimetic used to practice embodiments as provided herein can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. Routine experimentation will determine whether a mimetic is effective for practicing the invention, for example, if a mimetic composition is effective in specifically binding BRAF V600E protein. Methodologies detailed herein and others known to persons skilled in the art may be used to select or guide one to choose effective mimetic for practicing the compositions and/or methods of this invention.

Polypeptide mimetic compositions for practicing embodiments as provided herein can comprise any combination of non-natural structural components. In alternative aspects, mimetic compositions for practicing embodiments as provided herein can comprise one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds.

### Products of Manufacture and Kits

Provided are products of manufacture and kits for practicing methods as provided herein comprising chimeric or recombinant anti-human BRAF V600E Abs as provided herein; and optionally the products of manufacture and kits can further comprise some or all reagents needed to perform an IHC, and optionally can comprise instructions for practicing methods as provided herein.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions (PCRs) can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: IHC with Exemplary Abs

This example demonstrates that anti-BRAF V600E antibodies as provided herein, and methods and compositions incorporating these exemplary Abs, are effective and can be used to detect BRAF V600E. For example, the anti-BRAF V600E antibodies provided herein may be used to detect BRAF V600E in IHC on colorectal carcinomas (CRC) to differentiate between sporadic CRC and Lynch syndrome. Anti-BRAF V600E antibodies as provided herein also can be used to identify BRAF V600E positive melanomas, papillary thyroid carcinomas and hairy cell leukemia, and other cancers.

Tissues used for assessing the ability of the anti-BRAF V600E antibodies to detect BRAF V600E include various clinical tissues, including colorectal cancers, expressing different protein levels of BRAF V600E and cases that are negative for BRAF V600E. Clinical tissues (CT) also includes CRC cases with known BRAF V600E molecular status.

Level of background staining obtained with the exemplary BRAF V600E (the antibody comprising of heavy chain SEQ ID NO:2 and the light chain SEQ ID NO:6) Dako OMNIS™ IHC staining protocol was tested/evaluated on a tissue array including various tissues, including tonsil, liver, breast cancer, carcinoid, colon cancer, colon, melanomas, prostate, cerebellum, pancreas and kidney.

In addition, performance of the BRAF V600E antibody (chimeric or the antibody comprising heavy chain SEQ ID NO:2 and SEQ ID NO:6) was tested on colorectal carcinomas, melanomas, papillary thyroid carcinomas and hairy cell leukemia to support the efficacy of the antibody.

Clone Selection

Recombinant antibody specifically targeting the BRAF V600E mutation, represented by the peptide sequence of the amino acids 596 to 606 (GLATEKSRWSG) (SEQ ID NO:17) was obtained as follows: Vectors were synthesized by GENEART™ (Invitrogen) to contain the DNA sequence encoding the antibody heavy and light chains respectively. Cloning was made into pTT5™ vectors for transient expression in HEK293-6E cells.

Plasmids were generated encoding mouse IgG2a heavy chain version and mouse kappa1 light chain version for production of mouse IgGs. Additionally, plasmids were generated encoding chimeric antibody containing the constant regions from rabbit heavy and light chains linked to the mouse variable region, respectively. More specifically, the mouse heavy chain the constant domains 1, 2, and 3, and the hinge region were replaced with rabbit IgG heavy chain constant domains. For the light chain the mouse kappa constant domain was replaced with a rabbit light chain kappa1 constant domain. The remaining part of the respective heavy and light chains (variable domains) remained unaltered, having the same variable region as for the mouse antibody versions.

The plasmids were used to express recombinant mouse and chimeric mouse/rabbit antibodies, respectively.

The antibodies were quantified and used, non-purified, for testing in TIC in FLEX protocol (ENVISION™ FLEX System™, Dako). Specific staining of BRAF V600E was observed for both the recombinant mouse and recombinant chimeric antibodies.

Both antibodies were Protein A purified and tested again in IHC, both in FLEX+ and FLEX++ protocols on Dako OMNIS™.

Purification had given a slight improvement of signal to noise ratio, and therefore it was decided to proceed only with the Protein A purified antibodies. Furthermore, from this test the chimeric antibody version had an optimal performance concentration of 62.5 ng/mL and the mouse antibody of 0.5 μg/ml.

For all the clone selection part, protocol optimization, and DoE (Design o Experiment), the concentration of the antibody used has been determined by ELISA.

Protocols

Initial testing of the chimeric antibody was a 5-step 2-fold titration to narrow down an approximate optimal concentration (1×) for further testing. 1× was assumed to be around 60 ng/ml.

A titration of the performance benchmark antibody was performed on a target specific multiblock, and slides were evaluated by consensus of a group of trained observers.

Preliminary testing showed that the signal to noise ratio was improved by pretreatment in high pH and by a FLEX++ protocol. Further testing of primary antibody concentration, diluent and incubation time was performed. The best tradeoff between signal and noise was found to be a primary antibody concentration at 60 ng/ml diluted in S0809 and incubated for 20 min.

The double of the 1× concentration, 120 ng/ml, was tested on the full tissue package containing 33 positive specimens and 18 negative specimens. Slides were evaluated and results clearly showed that the primary antibody titer of 120 ng/mL was too high with excessive background staining and increased chances of false positive specimens. Slides was evaluated side by side with performance benchmark slides.

A small tissue package with 12 selected specimens was now stained with both 30 ng/ml and 60 ng/ml as primary antibody concentration, and from these tests it was concluded that the optimal BRAF V600E performance was somewhere in between the two tested concentrations. A compromise was made, and a full tissue package was stained with 45 ng/ml as primary antibody concentration.

This protocol (BRAF V600E, chimeric antibody diluted in S0809 to 45 ng/ml) seemed to have “good” signal to noise ratio with a low chance of detecting false positive specimens, as well as a strong concordance to Ventana's benchmark assay. All slides were evaluated side by side with performance benchmark slides.

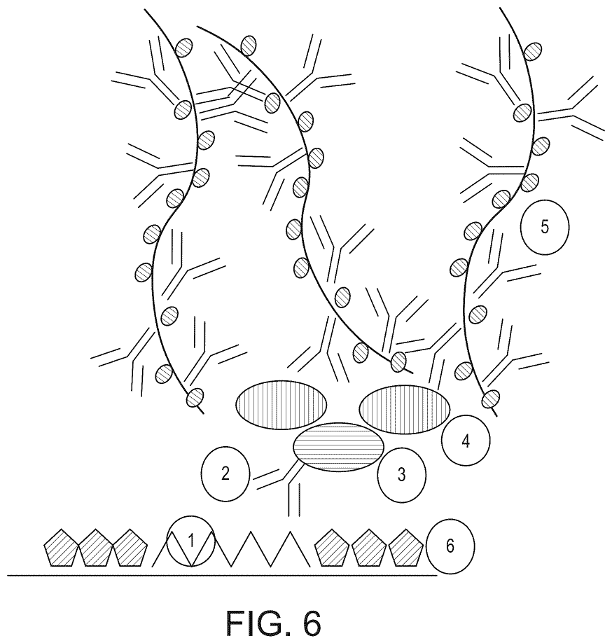

FIG. 6 schematically illustrates the principle of an exemplary IHC as provided herein using an exemplary anti-BRAF V600E antibody as provided herein, a so-called “the Dako FLEX++ system” on a Dako OMNIS™ instrument, as used for an TIC using the exemplary BRAF V600E antibody; the principle in the BRAF V600E FLEX++(Dako OMNIS™) IHC staining protocol is (as noted in the figure): 1. BRAF Antigen in sample, 2. Primary antibody (an exemplary anti-BRAF V600E antibody as provided herein), 3. Mouse Linker (or rabbit Linker), 4. Rabbit Linker (or mouse Linker), 5 ENVISION™ (or EnVision; a visualization reagent comprising an enzyme labeled polymer conjugated to secondary antibodies available from Agilent Technologies, Inc., Santa Clara, CA)), 6. DAB (visual brown reaction product at the antigen site).

Table 1, below, shows the performance of chimeric BRAF V600E antibody in colorectal cancer (CRC) specimens with optimal protocol on Dako OMNIS™. The BRAF V600E staining is localized to the cytoplasm. The staining intensity in CRC tumor cells varies from 0 to 2.75.

In rare cases weak nuclear staining was observed, which should be considered as non-specific. Furthermore, in some CRC specimens a weak grey/yellow cytoplasmic background staining was observed in tumor cells, normal epithelium, smooth muscle cells and other stromal cells. This weak grey/yellow staining was also observed with negative control reagent.

TABLE 1
Colorectal cancers stained with chimeric BRAF V600E antibody,
in optimal Dako OMNIS ™ protocol

| | | chimeric BRAF V600E, FLEX++ | | |
|---|---|---|---|---|
| | | Dako OMNIS ™ | | |
| | | | Tumor | |
| | Colorectal | | cells | BRAF |
| | cancers | Slide | staining | V600E |
| Comment | Block | no | intensity | status* |
| CT2 | 112146 | 1.2 | 0.875 | Positive |
| CT3 | 112146 | 1.3 | 2.75 | Positive |
| CT4 | 112146 | 1.4 | 1.625 | Positive |
| CT1 | 112147 | 2.1 | 2 | Positive |
| CT2 | 112147 | 2.2 | 1.375 | Positive |
| CT3 | 112147 | 2.3 | 2.75 | Positive |
| CT4 | 112147 | 2.4 | 1.875 | Positive |
| CT5 | 112147 | 2.5 | 1.875 | Positive |
| NA | 111903 | 4 | 2.5 | Positive |
| NA | 111905 | 5 | 0.875 | Positive |
| NA | 112072 | 6 | 1.5 | Positive |
| NA | 112073 | 7 | 0.5 | Positive |
| NA | 112144 | 8 | 1 | Positive |
| NA | 112145 | 9 | 1.5 | Positive |
| NA | 112150 | 10 | 0.875 | Positive |
| V600E | 114315 | 11 | 0.875 | Positive |
| mutation | | | | |
| status: positive | | | | |
| V600E | 114316 | 12 | 0.75 | Positive |
| mutation | | | | |
| status: positive | | | | |
| V600E | 114317 | 13 | 0.125 | Equivocal/ |
| mutation | | | | Negative |
| status: positive | | | | |
| NA | 30516 | 14 | 1.125 | Positive |
| NA | 43185 | 15 | 0.875 | Positive |
| NA | 119145 | 16 | 0.625 | Positive |
| NA | 44959 | 17 | 0.25 | Equivocal/ |
| | | | | positive |
| NA | 43519 | 19 | 0.75 | Positive |
| NA | 44960 | 20 | 0.375 | Positive |
| NA | 51078 | 21 | 0.5 | Positive |
| NA | 54448 | 22 | 0.75 | Positive |
| V600E | BRAF+ | 23 | 1 | Positive |
| mutation | Biopsy 1 | | | |
| status: positive | | | | |
| V600E | BRAF+ | 24 | 2 | Positive |
| mutation | Biopsy 2 | | | |
| status: positive | | | | |
| V600E | 1 BRAF+ | 34 | 0.625 | Positive |
| mutation | | | | |
| status: positive | | | | |
| V600E | 2 BRAF+ | 35 | 1.75 | Positive |
| mutation | | | | |
| status: positive | | | | |
| V600E | 3 BRAF+ | 36 | 0.625 | Positive |
| mutation | | | | |
| status: positive | | | | |
| V600E | 4 BRAF+ | 37 | 1 | Positive |
| mutation | | | | |
| status: positive | | | | |
| V600E | 5 BRAF+ | 38 | 1.25 | Positive |
| mutation | | | | |
| status: positive | | | | |
| NA | 34262 | 39 | 0 | Negative |
| NA | 50471 | 40 | 0 | Negative |
| NA | 50504 | 41 | 0 | Negative |
| NA | 50581 | 42 | 0.125 | Negative |
| NA | 50590 | 43 | 0.125 | Negative |
| NA | 50596 | 44 | 0-0.125 | Negative |
| NA | 51088 | 45 | 0.25 | Negative |
| NA | 65911 | 46 | 0.125 | Negative |
| NA | 66659 | 47 | 0 | Negative |
| NA | 68382 | 48 | 0.125 | Negative |
| NA | 72873 | 49 | 0.625 | Positive |
| NA | 51085 | 50 | 0.125 | Negative |
| V600E | BRAF− | 51 | 0.125 | Negative |
| mutation | | | | |
| status: negative | | | | |
| V600E | BRAF− | 52 | 0.125 | Negative |
| mutation | | | | |
| status: negative | | | | |
| V600E | 6 BRAF− | 53 | 0.125 | Negative |
| mutation | | | | |
| status: negative | | | | |
| V600E | 7 BRAF− | 54 | 0.125 | Negative |
| mutation | | | | |
| status: negative | | | | |
| V600E | 8 BRAF− | 55 | 0 | Negative |
| mutation | | | | |
| status: negative | | | | |
| V600E | 9 BRAF− | 56 | 0.125 | Negative |
| mutation | | | | |
| status: negative | | | | |
| V600E | 10 BRAF− | 57 | 0.25 | Equivocal/ |
| mutation | | | | Negative |
| status: negative | | | | |

*BRAF V600E clinical status was according to the FDA approved guidelines for BRAF V600E (clone VE1), Ventana Solution IFU: A clinical status of positive is assigned to cases with unequivocal cytoplasmic staining of any staining intensity in tumor cells above background. A clinical status of negative is assigned to cases with no or equivocal cytoplasmic staining in viable tumor cells. Nuclear staining, weak to strong of isolated viable tumor cells, and/or small tumor clusters should be considered negative.

### Results

CRC Stained with Chimeric BRAF V600E Antibody

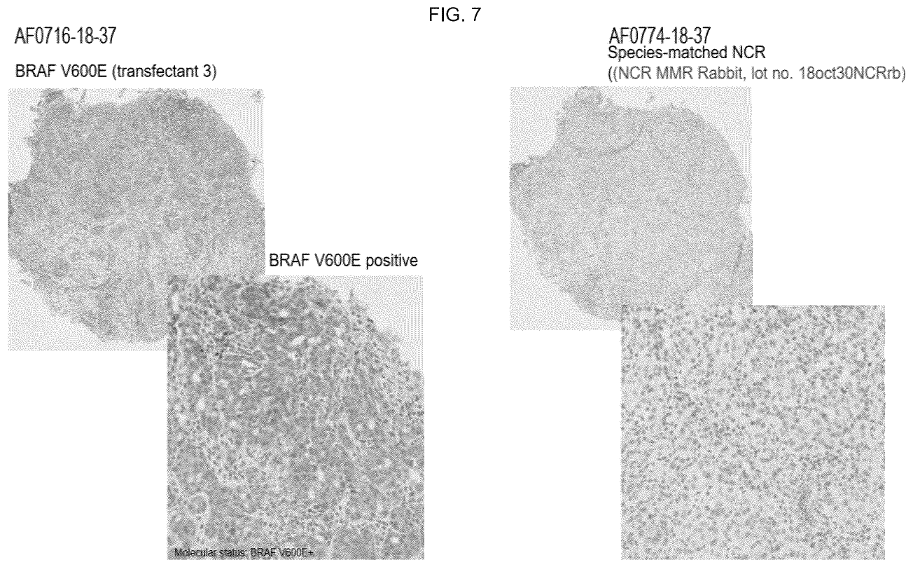

FIG. 7 illustrates an IHC of Sporadic Colorectal Cancer (CRC) (block 4 BRAF+ stained with the exemplary chimeric BRAF V600E antibody (45 ng/ml), where a weak to moderate cytoplasmic staining is observed in tumor cells. Note that a weak cytoplasmic background staining is observed with species-matched NCR.

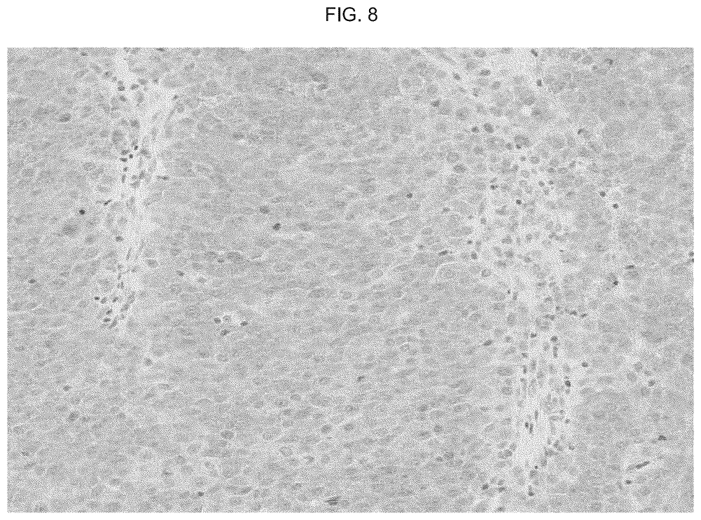

FIG. 8 illustrates a melanoma tissue block stained with the exemplary BRAF V600E (45 ng/ml), where a weak to moderate cytoplasmic staining is observed in tumor cells.

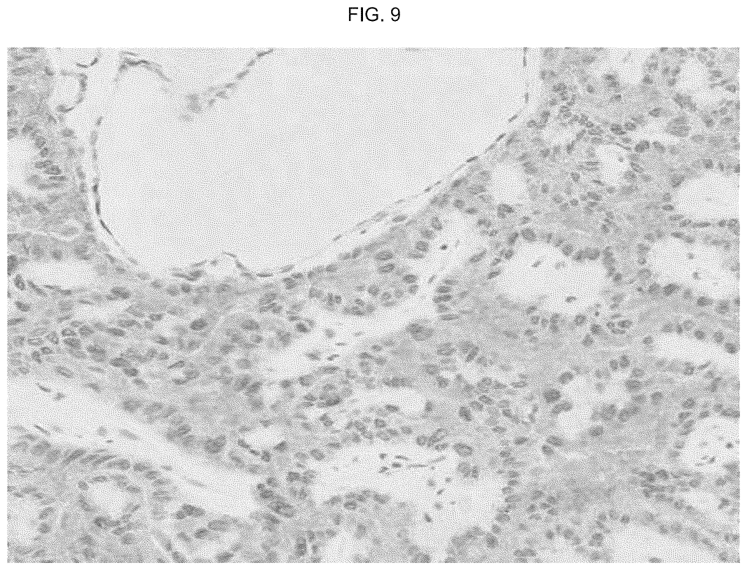

FIG. 9 illustrates a papillary thyroid carcinoma stained with chimeric BRAF V600E antibody (45 ng/ml), where a weak to moderate cytoplasmic staining is observed in tumor cells. Note that weak nuclear staining is observed in some tumor cells. Nuclear staining should be considered as non-specific staining.

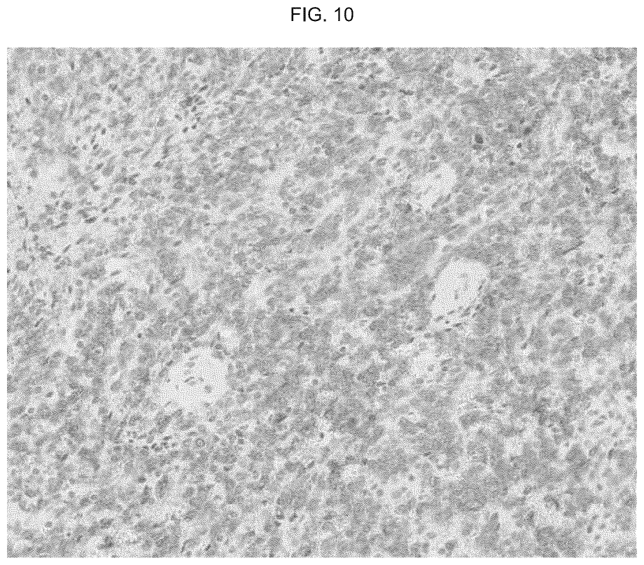

FIG. 10 illustrates a hairy cell leukemia stained with chimeric BRAF V600E antibody (45 ng/ml), where a weak to moderate cytoplasmic staining is observed in tumor cells. Comment: Artefacts, probably due to fixation, are present in this block.

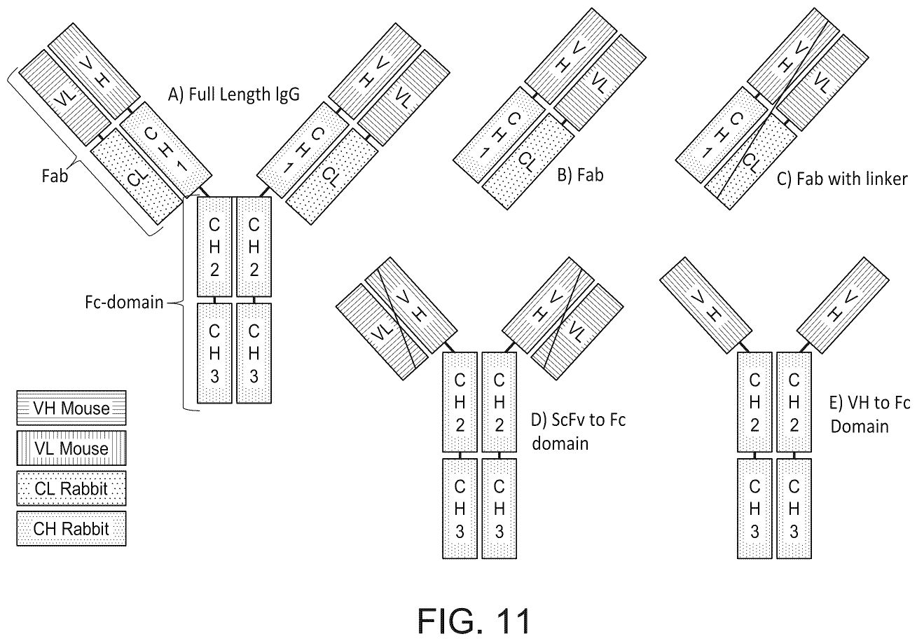

FIG. 11A-E schematically illustrate different versions of exemplary chimeric BRAF V600E antibodies as provided herein, for example, as described in claim 1: FIG. 11A illustrates the exemplary full length chimeric IgG (mouse variable (VL+VH) and rabbit constant domains (CL+CH1+CH2+CH3));

FIG. 11B illustrates the exemplary Fab fragment containing of mouse variable heavy (VH) and variable light (VL) chain domains, rabbit constant light chain domain (CL) and first heavy chain constant domain (CH1);

FIG. 11C illustrates the exemplary Fab fragment with linker, as FIG. 11B, but with linker covalently connecting the two chains;

FIG. 11D illustrates the exemplary ScFv antibody fragment in fusion to Fc domain; the variable heavy (VH) and light chain (VL) domains are covalently linked by a linker forming a ScFv in fusion to Fc domain, comprising or consisting of heavy chain constant 2 (CH2) and 3 (VH3) domains; and

FIG. 11E illustrates the exemplary single domain antibody fragment in fusion to Fc domain; here a variable heavy chain (VH) domain in fusion to the Fc domain, comprising or consisting of heavy chain constant 2 (CH2) and 3 (VH3) domains. Any of the above aspects and embodiments can be combined with any other aspect or embodiment as disclosed here in the Summary and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

### REFERENCES

1. Ascierto, P. A., et al., The role of BRAF V600 mutation in melanoma. J Transl Med, 2012. 10: p. 85.
2. Davies, H., et al., Mutations of the BRAF gene in human cancer. Nature, 2002. 417(6892): p. 949-54.
3. Rajagopalan, H., et al., Tumorigenesis: RAF/RAS oncogenes and mismatch-repair status. Nature, 2002. 418(6901): p. 934.
4. Brose, M. S., et al., BRAF and RAS mutations in human lung cancer and melanoma. Cancer Res, 2002. 62(23): p. 6997-7000.
5. Maldonado, J. L., et al., Determinants of BRAF mutations in primary melanomas. J Natl Cancer Inst, 2003. 95(24): p. 1878-90.
6. Lee, J. W., et al., BRAF mutations in non-Hodgkin's lymphoma. Br J Cancer, 2003. 89(10): p. 1958-60.
7. Tiacci, E., et al., BRAF mutations in hairy-cell leukemia. N Engl J Med, 2011. 364(24): p. 2305-15.
8. Kimura, E. T., et al., High prevalence of BRAF mutations in thyroid cancer: genetic evidence for constitutive activation of the RET/PTC-RAS-BRAF signaling pathway in papillary thyroid carcinoma. Cancer Res, 2003. 63(7): p. 1454-7.
9. Naoki, K., et al., Missense mutations of the BRAF gene in human lung adenocarcinoma. Cancer Res, 2002. 62(23): p. 7001-3.
10. Badalian-Very, G., et al., Recurrent BRAF mutations in Langerhans cell histiocytosis. Blood, 2010. 116(11): p. 1919-23.
11. Kurppa, K. J., et al., High frequency of BRAF V600E mutations in ameloblastoma. J Pathol, 2014. 232(5): p. 492-8.
12. Knobbe, C. B., et al., Mutation analysis of the Ras pathway genes NRAS, HRAS, KRAS and BRAF in glioblastomas. Acta Neuropathol, 2004. 108(6): p. 467-70.
13. Dougherty, M. J., et al., Activating mutations in BRAF characterize a spectrum of pediatric low-grade gliomas. Neuro Oncol, 2010. 12(7): p. 621-30.
14. Chapman, P. B., et al., Improved survival with vemurafenib in melanoma with BRAF V600E mutation. N Engl J Med, 2011. 364(26): p. 2507-16.
15. Young, K., et al., BRIM-1, -2 and -3 trials: improved survival with vemurafenib in metastatic melanoma patients with a BRAF(V600E) mutation. Future Oncol, 2012. 8(5): p. 499-507.
16. Flaherty, K. T., et al., Combined BRAF and MEK inhibition in melanoma with BRAF V600 mutations. N Engl J Med, 2012. 367(18): p. 1694-703.
17. Kim, K. B., et al., Phase II study of the MEK1/MEK2 inhibitor Trametinib in patients with metastatic BRAF-mutant cutaneous melanoma previously treated with or without a BRAF inhibitor. J Clin Oncol, 2013. 31(4): p. 482-9.
18. Planchard, D., et al., Dabrafenib plus trametinib inpatients with previously treated BRAF(V600E)-mutant metastatic non-small cell lung cancer: an open-label, multicentre phase 2 trial. Lancet Oncol, 2016. 17(7): p. 984-993.
19. Tissot, C., et al., Clinical characteristics and outcome of patients with lung cancer harboring BRAF mutations. Lung Cancer, 2016. 91: p. 23-8.
20. Corcoran, R. B., et al., Combined BRAF and MEK Inhibition With Dabrafenib and Trametinib in BRAF V600-Mutant Colorectal Cancer. J Clin Oncol, 2015. 33(34): p. 4023-31.
21. Corcoran, R. B., et al., Combined BRAF, EGFR, and MEK Inhibition in Patients with BRAF(V600E)-Mutant Colorectal Cancer. Cancer Discov, 2018. 8(4): p. 428-443.
22. Varadi, Z., et al., Effective BRAF inhibitor vemurafenib therapy in a 2-year-old patient with sequentially diagnosed Langerhans cell histiocytosis and Erdheim-Chester disease. Onco Targets Ther, 2017. 10: p. 521-526.
23. Del Bufalo, F., et al., BRAF V600E Inhibitor (Vemurafenib) for BRAF V600E Mutated Low Grade Gliomas. Front Oncol, 2018. 8: p. 526.
24. Wen, P., et al., ACTR-30. UPDATED EFFICACY AND SAFETY OF DABRAFENIB PLUS TRAMETINIB INPATIENTS WITH RECURREN TIRE FRACTORY BRAF V600E-MUTATED HIGH-GRADE GLIOMA (HGG) AND LOW-GRADE GLIOMA (LGG). Neuro-Oncology, 2019. 21(Supplement_6): p. vi19-vi20.
25. Kaye, F. J., et al., Clinical and radiographic response with combined BRAF-targeted therapy in stage 4 ameloblastoma. J Natl Cancer Inst, 2015. 107(1): p. 378.
26. Falini, B. and E. Tiacci, New treatment options in hairy cell leukemia with focus on BRAF inhibitors. Hematol Oncol, 2019. 37 Suppl 1: p. 30-37.
27. Petruzzellis, G., et al., Vemurafenib Treatment of Pleomorphic Xanthoastrocytoma in a Child With Down Syndrome. Front Oncol, 2019. 9: p. 277.
28. Haroche, J., et al., High prevalence of BRAF V600E mutations in Erdheim-Chester disease but not in other non-Langerhans cell histiocytoses. Blood, 2012. 120(13): p. 2700-3.

A number of embodiments of the invention have been described. Nevertheless, it can be understood that various modifications may be made without departing from the spirit and scope of the invention. Accordingly, other embodiments are within the scope of the following claims.

1: A chimeric or recombinant antibody (Ab) or antigen binding protein (ABP) capable of specifically binding a human BRAF V600E protein, selected from the group consisting of:
(a) a heterodimeric Ab or ABP comprising:
(i) a first and a second rabbit antibody Fc heavy chain, each comprising:
(1) a C2 or a C3 rabbit antibody Fc heavy chain constant domain, or portion thereof of sufficient length to allow binding by an antibody capable of specifically binding said portion,
wherein the first rabbit antibody Fc heavy chain constant domain, or a portion thereof, is bound to or associated with the second rabbit antibody Fc heavy chain constant domain, or a portion thereof,
(2) a C1 rabbit antibody heavy chain constant domain or portion thereof capable of associating with a rabbit antibody light chain constant region bound to the C2 or C3 rabbit antibody Fc heavy chain constant domain or portion thereof; and
(3) a heavy chain component or portion thereof of a mouse variable region antigen binding domain (ABD) or portion thereof capable of associating with a mouse light chain variable region ABD component or portion thereof, said heavy chain component of the mouse variable region antigen binding domain (ABD) or portion thereof being bound to the C1 rabbit antibody heavy chain constant domain or portion thereof,
wherein when the heavy chain component of the mouse variable region ABD or portion thereof is associated with a light chain component of a mouse variable region ABD or portion thereof, the combined heavy chain variable region component or portion thereof and light chain variable region component or portion thereof together can specifically bind to the human BRAF V600E protein, and
(ii) two light chains, each comprising:
(1) a C1 rabbit antibody light chain constant region or portion thereof,
wherein the rabbit antibody light chain constant region, or a portion thereof, is associated with, or is bound to, a C1 rabbit antibody heavy chain constant domain or portion thereof, and
(2) the light chain component of a mouse variable region antigen binding domain (ABD) or portion thereof in combination with, or is bound to or associated with, the mouse heavy chain variable region ABD component or portion thereof;
(b) a Fab heterodimer comprising:
(i) a first chain comprising:
(1) a C1 rabbit antibody heavy chain constant domain or portion thereof capable of associating with or binding to a C1 rabbit antibody light chain constant region or portion thereof, bound to
(2) a heavy chain component of a mouse variable region antigen binding domain (ABD) or portion thereof capable of associating with or binding to a mouse light chain variable region ABD component or portion thereof,
wherein the C1 rabbit antibody light chain constant region or portion thereof is associated with, or is bound to, the C1 rabbit antibody heavy chain constant domain; and
(ii) a second chain comprising:
(1) a C1 rabbit antibody light chain constant region or portion thereof capable of associating with or binding to a C1 rabbit antibody heavy chain constant domain or portion thereof, bound to
(2) a light chain component of a mouse variable region antigen binding domain (ABD) or portion thereof capable of associating with or binding to a mouse heavy chain variable region ABD component or portion thereof,
wherein when the heavy chain component of the mouse variable region ABD is combined with, or is bound to, the light chain component of the mouse ABD, and the combined or bound heavy chain variable region ABD component and light chain variable region ABD component can specifically bind to the human BRAF V600E protein;
(c) a heterodimeric Ab or ABP comprising two Fab heterodimers of (b), wherein the C1 rabbit antibody heavy chain constant domain or portion thereof and the C1 rabbit antibody light chain constant region or portion thereof are joined by a linker;
(d) a dimeric ABP comprising:
(i) a first and a second heavy chain, each comprising:
(1) a C2 or a C3 rabbit antibody Fc heavy chain constant domain, or a portion thereof of sufficient length to allow binding by an antibody capable of specifically binding said portion,
wherein the C2 or a C3 rabbit antibody Fc heavy chain constant domain, or a portion thereof, of the first heavy chain is associated with or bound to the second C2 or C3 rabbit antibody Fc heavy chain constant domain, or a portion thereof,
(2) a heavy chain component of a mouse variable region antigen binding domain (ABD) or portion thereof capable of associating with and bound to a mouse light chain variable region ABD component or portion thereof,
wherein when the heavy chain component of the mouse variable region ABD or portion thereof is associated with and bound to the light chain component of the mouse variable region ABD or portion thereof, and the combined heavy chain variable component or portion thereof and light chain variable component or portion thereof can specifically bind to the human BRAF V600E protein, and
(ii) a light chain comprising: the light chain component of a mouse variable region antigen binding domain (ABD) or portion thereof bound to the mouse heavy chain variable region ABD component or portion thereof; or
(e) a dimeric ABP comprising:
(i) a first and a second heavy chain, each comprising:
(1) a heavy chain component of a mouse variable region antigen binding domain (ABD) or portion thereof capable of specifically bind to the human BRAF V600E protein, or, a light chain component of a mouse variable region antigen binding domain (ABD) or portion thereof capable of specifically bind to the human BRAF V600E protein, and
(2) a C2 or C3 rabbit antibody Fc heavy chain constant domain, or a portion thereof of sufficient length to allow binding by an antibody capable of specifically binding said portion,
wherein the C2 or C3 rabbit antibody Fc heavy chain constant domain, or portion thereof, of the first heavy chain is associated with or bound to the other rabbit antibody C2 or C3 Fc heavy chain constant domain, or portion thereof of the second heavy chain.

2: The chimeric or recombinant antibody (Ab) or antigen binding protein (ABP) of claim 1, wherein:
the first and the second rabbit antibody Fc heavy chain of (a)(i)(1) each comprises both a C2 rabbit antibody Fc heavy chain constant domain, or portion thereof, and a C3 rabbit antibody Fc heavy chain constant domain, or portion thereof;
in embodiment (a)(ii)(1) the first rabbit antibody Fc heavy chain constant domain, or portion thereof, is covalently or non-covalently bound to or associated with the second rabbit antibody Fc heavy chain constant domain, or portion thereof;
in embodiment (a)(ii)(2) the rabbit antibody light chain constant region, or portion thereof, is covalently or non-covalently associated with, or is bound to, the C1 rabbit antibody heavy chain constant domain;
in embodiment (b) the C1 rabbit antibody light chain constant region or portion thereof is covalently or non-covalently associated with, or is bound to, the C1 rabbit antibody heavy chain constant domain or portion thereof;
in embodiment (b) the light chain component of the mouse variable region antigen binding domain (ABD) or portion thereof is covalently or non-covalently capable of associating with or binding to a mouse heavy chain variable region ABD component or portion thereof;
in embodiment (c) the C1 rabbit antibody heavy chain constant domain or portion thereof and the C1 rabbit antibody light chain constant region or portion thereof are joined by a chemical or a peptide linker;
in embodiment (d) both the first and the second heavy chain each comprise both a C2 rabbit antibody Fc heavy chain constant domain, or portion thereof, and a C3 rabbit antibody Fc heavy chain constant domain, or portion thereof;
in embodiment (d) the C2 or the C3 rabbit antibody Fc heavy chain constant domain, or a portion thereof, of the first heavy chain is covalently or non-covalently associated with or bound to the second C2 or C3 rabbit antibody Fc heavy chain constant domain, or a portion thereof;
in embodiment (d) the light chain component of the mouse variable region antigen binding domain (ABD) or portion thereof is covalently or non-covalently bound to the mouse heavy chain variable region ABD component or portion thereof;
in embodiment (e) the first and the second heavy chain each comprises both a C2 and a C3 rabbit antibody Fc heavy chain constant domain, or a portion thereof of sufficient length to allow binding by an antibody capable of specifically binding said portion; or
in embodiment (e) the C2 or C3 rabbit antibody Fc heavy chain constant domain, or portion thereof, of the first heavy chain is covalently or non-covalently associated with or bound to the rabbit antibody C2 or C3 Fc heavy chain constant domain, or portion thereof of the second heavy chain.

3: The chimeric or recombinant antibody (Ab) or antigen binding protein (ABP) of claim 1, wherein:
the rabbit antibody heavy chain constant region is a rabbit IgG antibody heavy chain constant region;
the rabbit antibody light chain constant region is a rabbit IgG antibody light chain constant region;
the rabbit antibody heavy chain constant region is a rabbit IgG antibody heavy chain constant region and the rabbit antibody light chain constant region is a rabbit IgG antibody light chain constant region;
the rabbit antibody light chain IgG constant region comprises a rabbit antibody kappa1, Kappa2, or a rabbit Lambda light chain constant region;
the rabbit lambda light chain constant region comprises rabbit lambda light chain 1, 2, 3, 4, 5 or 6;
the heavy chain component of the mouse ABD comprises at least one of: a complementarity determining region (CDR) comprising SEQ ID NO:9 having one or more amino acid substitutions, additions (insertions) or deletions, a CDR comprising SEQ ID NO:10 having one or more amino acid substitutions, additions (insertions) or deletions, and a CDR comprising SEQ ID NO:11 having one or more amino acid substitutions, additions (insertions) or deletions, and the chimeric or recombinant Ab or ABD is capable of specifically binding a human BRAF V600E protein;
the chimeric or recombinant Ab or ABD is capable of specifically binding a human BRAF V600E protein with greater or lesser affinity than the sequence without the at least one conservative amino acid substitution; or
the heavy chain component of the mouse ABD comprises at least one of: a complementarity determining region (CDR) having an amino acid sequence having at least about 70%, 75%, 80%, 85%, 90%, 95%, 98% or complete sequence identity to SEQ ID NO:9, a CDR having an amino acid sequence having at least about 70%, 75%, 80%, 85%, 90%, 95%, 98% or complete sequence identity to SEQ ID NO:10, and a CDR having an amino acid sequence having at least about 70%, 75%, 80%, 85%, 90%, 95%, 98% or complete sequence identity to SEQ ID NO:11, and the chimeric or recombinant Ab or ABD is capable of specifically binding a human BRAF V600E protein.

4: The chimeric or recombinant antibody (Ab) or antigen binding protein (ABP) of claim 1, wherein:
the heavy chain component of the mouse ABD comprises SEQ ID NO:9, SEQ ID NO:10 and SEQ ID NO:11;
the light chain component of the mouse ABD comprises at least one of: a complementarity determining region (CDR) comprising SEQ ID NO:12 having one or more amino acid substitutions, additions, insertions or deletions, a CDR comprising SEQ ID NO:13 having one or more amino acid substitutions, additions (insertions) or deletions and a CDR comprising SEQ ID NO:14 having one or more amino acid substitutions, additions, insertions or deletions, and the chimeric or recombinant Ab or ABD is capable of specifically binding a human BRAF V600E protein;
the chimeric or recombinant Ab or ABD is capable of specifically binding a human BRAF V600E protein with greater or lesser affinity than the sequence without the at least one conservative amino acid substitution;
the light chain component of the mouse ABD comprises at least one of: a complementarity determining region (CDR) having an amino acid sequence having at least about 70%, 75%, 80%, 85%, 90%, 95%, 98% or complete sequence identity to SEQ ID NO:12, a CDR having an amino acid sequence having at least about 70%, 75%, 80%, 85%, 90%, 95%, 98% or complete sequence identity to SEQ ID NO:13, and a CDR having an amino acid sequence having at least about 70%, 75%, 80%, 85%, 90%, 95%, 98% or complete sequence identity to SEQ ID NO:14, and the chimeric or recombinant Ab or ABD is capable of specifically binding a human BRAF V600E protein;
the light chain component of the mouse ABD comprises SEQ ID NO:12, SEQ ID NO:13 and SEQ ID NO:14;
the chimeric or recombinant antibody comprises: a heavy chain comprising a sequence as set forth in SEQ ID NO:1 and a light chain as set forth in SEQ ID NO:5;
or, a heavy chain comprising a sequence as set forth in SEQ ID NO:2 and a light chain as set forth in SEQ ID NO:6;
each heavy chain comprises two rabbit Fc heavy chain constant region domains;
the linker comprises: a polyglycine linker, or a (GlyGlyGlySer)x, wherein x is 2, 3 or 4;
the chimeric or recombinant antibody (Ab) or antigen binding protein (ABP) further comprises a heterologous protein or peptide, or the heterologous protein or peptide comprises a peptide or polypeptide tag or detectable moiety;
the peptide or polypeptide tag comprises an epitope tag, or the epitope peptide or polypeptide tag comprises a Myc-tag, a Human influenza hemagglutinin (HA) tag, FLAG-tag, GST-tag, a polyhistidine (His) or a 6×His, V5-tag or OLLAS (E. coli OmpF Linker and mouse Langerin fusion Sequence);
the chimeric or recombinant Ab or ABP is bound to a detectable agent or a binding moiety, or the chimeric or recombinant Ab or ABP is covalently bound to the detectable agent or the binding moiety; or
the detectable agent or binding moiety comprises a biotin, a fluorescent or chemiluminescent label, a fluorophore, sulfoindocyanine, nile red, rhodamine, perylene, fluorenyl, coumarin, 7-methoxycoumarin (Mca), dabcyl, [2-(4-nitro-2,1,3-benzoxadiazol-7-yl)aminoethyl]trimethylammonium (NBD), Nile blue, Tamra, boron-dipyrromethene (BODIPY), or derivatives thereof, a dye, a radioisotope, a quantum dot or photoluminescent aqueous nanocrystal, a hapten, or an antibody binding epitope or domain, or the detectable agent or binding moiety comprises a fluorophore comprising a dansyl, a fluorescein, a carboxyfluorescein (FAM) or a 6-FAM moiety, or the dye is or comprises a cyanine dye, a Cy3 or a Cy5, or the detectable agent or binding moiety comprises a a biotin, a theophylline, a digoxigenin, a carborane, a fluorescein or a bromodeoxyuridine moiety.

5: A chimeric or recombinant nucleic acid encoding a chimeric or recombinant antibody of claim 1.

6: The chimeric or recombinant nucleic acid of claim 5, wherein:
the chimeric or recombinant nucleic acid further comprises and is operatively linked to a transcriptional regulatory element; or
the transcriptional regulatory element comprises a promoter, or the transcriptional regulatory element comprises an inducible promoter or a constitutive promoter.

7: An expression cassette, vector, recombinant virus, artificial chromosome, cosmid, phagemid or plasmid comprising a chimeric or recombinant nucleic acid of claim 5.

8: A cell comprising a chimeric or recombinant antibody of claim, or the cell is a bacterial, fungal, mammalian, yeast, insect or plant cell.

9: A method for detecting the presence of a human BRAF V600E protein in a cell or a tissue or organ or a portion of any of the foregoing comprising contacting the cell, tissue or organ or a portion of any of the foregoing with a chimeric or recombinant antibody of claim 1.

10: The method of claim 9, wherein:
the contacting comprises use of an immunohistochemistry (IHC) assay;
the method further comprises contacting the chimeric or recombinant antibody, with a detectable agent to indicate or signal binding, or non-binding, of said chimeric or recombinant antibody to said BRAF V600E protein;
said detectable agent comprises an antibody or antigen binding moiety which binds to said rabbit constant region or portion thereof; or
the detectable agent comprises a chromogenic substrate bound to the antibody or antigen binding moiety;
the chromogenic substrate comprises 3,3′-diaminobenzidine (DAB), 3,3′,5,5′-tetramethylbenzidine (TMB) or 2,2′-azino-bis(3-ethylbenzothiazoline-6-sulfonic acid) (ABTS);
the method further comprises the addition of a peroxidase enzyme and hydrogen peroxidase or equivalent oxidizing agent to oxidize the chromogenic substrate, or the method further comprises the addition of a horse radish peroxidase; or
the method further comprises determining whether the cell, tissue, organ or portion of any of the foregoing has reduced mismatch repair gene MLH1 expression or activity or has no mismatch repair gene MLH1 expression or activity.

11: A method for determining whether a colorectal cancer (CRC) in a subject is the result of a sporadic mutation or probable Lynch syndrome comprising:
(a) determining whether the subject has reduced MLH1 expression or activity or has no MLH1 expression or activity; and
(b) if the subject has reduced MLH1 expression or activity or has no MLH1 expression or activity, determining whether the subject is expressing the BRAF V600E protein using a chimeric or recombinant antibody of claim 1,
wherein the colorectal cancer is determined to be the result of:
(i) a sporadic mutation in the MLH1 gene if the subject expresses the BRAF V600E protein, or
(ii) probable Lynch syndrome if the subject does not express the BRAF V600E protein.

12: A method for differentiating between Sporadic Colorectal Cancer (CRC) and Lynch syndrome in a CRC patient, or to identify probable Lynch syndrome in a CRC patient having loss of DNA mismatch repair gene MLH1, comprising;
(a) conducting an immunohistochemistry assay using a chimeric or recombinant antibody of claim 1, to detect the expression or presence of a human BRAF V600E protein in a cell or tissue sample from the CRC patient; or
(b) the method of (a) further comprises evaluating cell or a tissue using antibody which specifically binds to an MLH1 protein, and if the cell or tissue is found to be MLH1 deficient, the cell or tissue is evaluated for the presence of BRAF V600E protein, and if the cell or tissue is positive for staining for BRAF V600E protein the CRC is sporadic CRC, and if the cell or tissue if negative for staining for BRAF V600E protein the CRC is associated with Lynch syndrome.

13. (canceled)

14: A method for detecting or diagnosing a cancer, wherein the method comprises:
(a) detection of expression or presence of a human BRAF V600E protein in a cell, tissue or organ sample, using a chimeric or recombinant antibody of any of claims 1 to 4, to detect the expression or presence of the human BRAF V600E protein in the cell, tissue or organ sample, or the detection comprises conducting an immunohistochemistry (IHC) assay,
and detecting the expression or presence of the human BRAF V600E protein in the cell, tissue or organ sample detects or diagnoses the cancer; or
(b) the method of (a), wherein:
the cancer is a colorectal cancer, a melanoma, non-Hodgkin lymphoma, hairy cell leukemia, papillary thyroid carcinoma, a lung cancer, Langerhans cell histiocytosis, ameloblastoma, a brain tumor, or hereditary nonpolyposis colorectal cancer (HNPCC);
the melanoma is a malignant melanoma;
the brain tumor is a glioblastoma or pleomorphic xanthoastrocytoma;
the lung cancer is an adenocarcinoma of the lung or a non-small-cell lung cancer; or
the method comprises detection of expression or presence of a human BRAF V600E protein in a cell, tissue or organ sample from an individual in need thereof, or the detection comprises conducting an immunohistochemistry (IHC) assay.

15. (canceled)

16: A method for treating, ameliorating or preventing a cancer comprising first detecting or diagnosing the cancer using a method of claim 14, followed by treatment of the individual in need thereof for the treatment, amelioration or prevention of the cancer.

17: A method for detecting or diagnosing an inflammatory disease, comprising:
(a) detecting expression or presence of a human BRAF V600E protein in a cell, tissue or organ sample, using of chimeric or recombinant antibody claim 1, to detect the expression or presence of the human BRAF V600E protein in the cell, tissue or organ sample,
and detecting the expression or presence of the human BRAF V600E protein in the tissue or organ sample detects or diagnoses the inflammatory disease; or
(b) the method of (a) wherein;
the inflammatory disease is Erdheim-Chester disease or polyostotic sclerosing histiocytosis;
the method comprises detection of expression or presence of a human BRAF V600E protein in a cell, tissue or organ sample from an individual in need thereof; or
the detection comprises conducting an immunohistochemistry (IHC) assay.

18. (canceled)

19: A method for treating, ameliorating or preventing an inflammatory disease comprising first detecting or diagnosing the inflammatory disease using a method claim 17, followed by treatment of the individual in need thereof for the treatment, amelioration or prevention of the inflammatory disease.

20. (canceled)

21: A kit comprising a chimeric or recombinant antibody claim 1, or the kit comprises components needed for an immunohistochemistry (IHC) assay.

22. A chimeric or recombinant antigen binding protein (ABP) capable of specifically binding a human BRAF V600E protein comprising:
(a) a rabbit antibody (Ab) heavy chain Fc region or portion thereof which is capable of specifically binding to a detectable secondary antibody or portion thereof; and
an antigen (Ag) binding region or an Ag-binding portion thereof comprising an Ab variable region from a species other than rabbit,
wherein the variable region specifically binds to the human BRAF V600E protein; or
(b) the chimeric or recombinant antigen binding protein of (a), wherein:
the antigen binding region comprises a Fab region;
the Fab region comprises a heavy chain region comprising a rabbit H C1 region fused to a mouse VH region and wherein said heavy chain region is associated with or bound to a light chain region comprising a rabbit L C1 region fused to a mouse VL region;
the antigen binding region comprises a ScFv region;
the ScFv region comprises a mouse VH region fused to a mouse VL region; or
the antigen binding region comprises a mouse VH region.

23. (canceled)

24: A chimeric or recombinant antigen binding protein comprising two Fab regions joined by a linker, wherein each Fab region comprises a heavy chain region comprising a rabbit H C1 region fused to a mouse VH region and wherein said heavy chain region is associated with a light chain region comprising a rabbit L C1 region fused to a mouse VL region.


SEQUENCE LISTING (17 sequences)

SEQ ID NO: 1 - protein, 456 aa, Artificial Sequence
  Synthetic Heavy chain with signal peptide
METGLRWLLLVAVLKGVQCQAYLQQSGAELVRPGASVKMSCKASGYTFTSYNMHWIKQTPRQGLEWIGAIYPGNGDTSYNQKFKGKATLTVDRSSNTAYMQLSSLTSEDSAVYFCARDGISPWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 2 - protein, 437 aa, Artificial Sequence
  Synthetic Heavy chain without signal peptide
QAYLQQSGAELVRPGASVKMSCKASGYTFTSYNMHWIKQTPRQGLEWIGAIYPGNGDTSYNQKFKGKATLTVDRSSNTAYMQLSSLTSEDSAVYFCARDGISPWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 3 - protein, 19 aa, Artificial Sequence
  Synthetic rabbit IgG heavy chain signal peptide
METGLRWLLLVAVLKGVQC

SEQ ID NO: 4 - protein, 323 aa, Artificial Sequence
  Synthetic the rabbit IgG heavy chain constant domain
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 5 - protein, 233 aa, Artificial Sequence
  Synthetic Light chain with signal peptide
MDTRAPTQLLGLLLLWLPGATFDIKMTQSPSSMYASLGERVTITCKASQDINRYLSWFQQKPGKSPKTLIYRANRVLDGVPSRFSGSGSGQDYSLTISSLESEDMGIYYCLHYDEYPYTYGGGTKLEIKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 6 - protein, 211 aa, Artificial Sequence
  Synthetic Light chain without signal peptide
DIKMTQSPSSMYASLGERVTITCKASQDINRYLSWFQQKPGKSPKTLIYRANRVLDGVPSRFSGSGSGQDYSLTISSLESEDMGIYYCLHYDEYPYTYGGGTKLEIKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 7 - protein, 22 aa, Artificial Sequence
  Synthetic the rabbit IgG heavy chain signal peptide
MDTRAPTQLLGLLLLWLPGATF

SEQ ID NO: 8 - protein, 104 aa, Artificial Sequence
  Synthetic rabbit kappa1 light chain constant domain
GDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 9 - protein, 8 aa, Artificial Sequence
  Synthetic CDR1
GYTFTSYN

SEQ ID NO: 10 - protein, 8 aa, Artificial Sequence
  Synthetic CDR2
IYPGNGDT

SEQ ID NO: 11 - protein, 7 aa, Artificial Sequence
  Synthetic CDR3
ARDGISP

SEQ ID NO: 12 - protein, 6 aa, Artificial Sequence
  Synthetic CDR1
QDINRY

SEQ ID NO: 13 - protein, 3 aa, Artificial Sequence
  Synthetic CDR2
RAN

SEQ ID NO: 14 - protein, 9 aa, Artificial Sequence
  Synthetic CDR3
LHYDEYPYT

SEQ ID NO: 15 - protein, 16 aa, Artificial Sequence
  Synthetic polyglycine linker
  SITE (9)..(16): “Gly Gly Gly Ser” may or may not be present
GGGSGGGSGGGSGGGS

SEQ ID NO: 16 - protein, 6 aa, Artificial Sequence
  Synthetic 6xHis tag
HHHHHH

SEQ ID NO: 17 - protein, 11 aa, Artificial Sequence
  Synthetic Recombinant antibody specifically targeting the BRAF V600E mutation
GLATEKSRWSG